(19) United States
(12) Patent Application Publication
(10) Pub. No.: US 2026/0023075 A1
(43) Pub. Date: Jan. 22, 2026

(54) ANTIBODIES AND BIOSENSORS FOR DETECTION OF PFAS COMPOUNDS

(71) Applicant: FREDsense Technologies Corp., Calgary (CA)

(72) Inventors: Julia Guegueniat, Calgary (CA); Emily Candice Hicks, Calgary (CA); Robert Mayall, Calgary (CA); Margaret Renaud-Young, Calgary (CA)

(73) Assignee: FREDsense Technologies Corp., Calgary, AB (CA)

(21) Appl. No.: 19/339,721

(22) Filed: Sep. 25, 2025

Related U.S. Application Data
(63) Continuation of application No. PCT/CA2024/050395, filed on Mar. 28, 2024.
(60) Provisional application No. 63/455,650, filed on Mar. 30, 2023.

(51) Int. Cl. G01N 33/543; C07K 16/44; G01N 33/53

(52) U.S. Cl. CPC G01N 33/54386; C07K 16/44; G01N 33/5308; C07K 2317/33; C07K 2317/569

(57) ABSTRACT

Antibodies and antibody fragments that bind to polyfluoroalkyl and perfluoroalkyl substances (PFAS) are described for use in biosensors for detection of PFAS compounds in environmental samples. Detectors incorporating the biosensors and kits for use with the detectors are also described.

### CROSS-REFERENCE TO RELATED APPLICATIONS

This application, filed under 35 U.S.C § 111 (a), is a continuation of PCT Application No. PCT/CA2024/050395, filed on Mar. 28, 2024, which claims the benefit of priority to U.S. Provisional Patent Application No. 63/455,650, filed on Mar. 30, 2023. The above-referenced patent applications are herein incorporated by reference in their entireties.

### SEQUENCE LISTING

The present application is being filed together with an XML Sequence Listing in electronic format for compliance with WIPO Sequence Listing Standard ST.26. The Sequence Listing file, entitled “10383-114885-01.xml”, was created on Sep. 19, 2025, and is 141,958 bytes in size. The information in electronic format of the Sequence Listing is incorporated herein by reference in its entirety.

### TECHNICAL FIELD

The technology relates to antibodies and antibody fragments that bind to polyfluoroalkyl and perfluoroalkyl substances (PFAS) and detectors which use the antibodies and antibody fragments which are used to detect PFAS compounds in environmental samples.

### BACKGROUND

Polyfluoroalkyl and perfluoroalkyl substances (PFAS) make up a large group of persistent anthropogenic chemicals used in industrial processes and commercial products over the past 60 years. Widespread use and extreme resistance to degradation have resulted in the ubiquitous presence of these compounds in the environment. The 2011-2012 U.S. National Health and Nutrition Examination Survey reported detectable serum PFAS concentrations in virtually all individuals (97%). Human PFAS exposure has been linked to cancer, elevated cholesterol, obesity, immune suppression, and endocrine disruption. Health concerns have prompted manufacturers in Europe and North America to phase out production of some long-chain PFAS. Limited available data suggest widespread exposure to replacement (short-chain) PFASs may also adversely affect human health. Human PFAS exposure includes dietary sources, household dust, air, and drinking water. Exposure from drinking water is a serious concern because of the high aqueous solubility of many PFASs. Relatively low PFAS concentrations can lead to elevated exposures in the general population. Elevated PFAS concentrations in U.S. drinking water have been reported in numerous regions, especially near industrial sites that produce or use them (Hu et al., Environ. Sci. Technol. Lett. 2016, 3, 10, 344-350, incorporated herein by reference in its entirety).

Current technologies for detecting PFAS can reliably quantify about 50 specific PFAS, but these technologies are unable to detect or quantify the thousands of other PFAS known to exist. A common conventional method for detection of PFAS compounds is LC-MS/MS, which is expensive and time consuming.

There is a need for improved technologies for detection of PFAS compounds, particularly detector systems which can be operated outside of laboratories.

### SUMMARY

According to one embodiment, there is provided an antibody or antibody fragment which binds at least to perfluorononanoic acid (PFNA) including: a CDR1 amino acid sequence having at least about 80% sequence identity to a sequence selected from the group consisting of SEQ ID NOs: 55, 60, 64 and 67; a CDR2 amino acid sequence having at least about 80% sequence identity to a sequence selected from the group consisting of SEQ ID NOs: 56, 61, 65 and 68; and a CDR3 amino acid sequence having at least about 80% sequence identity to a sequence selected from the group consisting of SEQ ID NOs: 57, 62, 66 and 69.

The antibody or antibody fragment may include a CD4 amino acid sequence having at least about 80% sequence identity to a sequence selected from the group consisting of SEQ ID NOs: 58, 59, 63 and 70.

The antibody or antibody fragment may include a VHH antibody sequence having at least about 80% sequence identity with a sequence selected from the group consisting of SEQ ID NOs: 1-8.

According to another embodiment, there is provided an antibody or antibody fragment which binds at least to perfluorooctane sulfonate (PFOS), including: a CDR1 amino acid sequence having at least about 80% sequence identity to a sequence selected from the group consisting of SEQ ID NOs: 71 and 75; a CDR2 amino acid sequence having at least about 80% sequence identity to a sequence selected from the group consisting of SEQ ID NOs: 72 and 76; and a CDR3 amino acid sequence having at least about 80% sequence identity to a sequence selected from the group consisting of SEQ ID NOs: 73 and 77.

The antibody or antibody fragment may include a CD4 amino acid sequence having at least about 80% sequence identity to a sequence selected from the group consisting of SEQ ID NOs: 74 and 78.

The antibody or antibody fragment may include a VHH antibody sequence having at least about 80% sequence identity with a sequence selected from the group consisting of SEQ ID NOs: 9 and 10.

According to another embodiment, there is provided an antibody or antibody fragment which binds at least to perfluorobutane sulfonate (PFBS) including: a CDR1 amino acid sequence having at least about 80% sequence identity to a sequence selected from the group consisting of SEQ ID NOs: 55 and 79; a CDR2 amino acid sequence having at least about 80% sequence identity to a sequence selected from the group consisting of SEQ ID NOs: 56 and 80; and a CDR3 amino acid sequence having at least about 80% sequence identity to a sequence selected from the group consisting of SEQ ID NOs: 57 and 81.

The antibody or antibody fragment may include a CD4 amino acid sequence having at least about 80% sequence identity to a sequence selected from the group consisting of SEQ ID NOs: 58 and 59.

The antibody or antibody fragment may include a VHH antibody sequence having at least about 80% sequence identity with a sequence selected from the group consisting of SEQ ID NOs: 11-13.

According to another embodiment, there is provided an antibody or antibody fragment which binds at least to perfluorooctanoic acid (PFOA), including: a CDR1 amino acid sequence having at least about 80% sequence identity to a sequence selected from the group consisting of SEQ ID NOs: 55, 82, 86, 90, 93, 131, 135, 139 and 143; a CDR2 amino acid sequence having at least about 80% sequence identity to a sequence selected from the group consisting of SEQ ID NOs: 56, 83, 87, 91, 94, 132, 136, 140 and 144; and a CDR3 amino acid sequence having at least about 80% sequence identity to a sequence selected from the group consisting of SEQ ID NOs: 57, 84, 88, 92, 95, 133, 137, 141 and 145.

The antibody or antibody fragment may include a CD4 amino acid sequence having at least about 80% sequence identity to a sequence selected from the group consisting of SEQ ID NOs: 58, 59, 85, 89, 134, 138 and 142.

The antibody or antibody fragment may include a VHH antibody sequence having at least about 80% sequence identity with a sequence selected from the group consisting of SEQ ID NOs: 14-18.

According to another embodiment, there is provided an antibody or antibody fragment which binds at least to perfluorohexane sulfonate (PFHxS), including: a CDR1 amino acid sequence having at least about 80% sequence identity to a sequence selected from the group consisting of SEQ ID NOs: 96, 100, 104, 107, 110 and 114; a CDR2 amino acid sequence having at least about 80% sequence identity to a sequence selected from the group consisting of SEQ ID NOs: 97, 101, 105, 108, 111; and 115; and a CDR3 amino acid sequence having at least about 80% sequence identity to a sequence selected from the group consisting of SEQ ID NOs: 98, 102, 106, 109, 112 and 116.

The antibody or antibody fragment may include a CD4 amino acid sequence having at least about 80% sequence identity to a sequence selected from the group consisting of SEQ ID NOs: 59, 74, 99, 103, and 113.

The antibody or antibody fragment may include a VHH antibody sequence having at least about 80% sequence identity with a sequence selected from the group consisting of SEQ ID NOs: 19-24.

According to another embodiment, there is provided an antibody or antibody fragment which binds at least to hexafluoropropylene oxide dimer acid (GenX) including: a CDR1 amino acid sequence having at least about 80% sequence identity to SEQ ID NO: 55; a CDR2 amino acid sequence having at least about 80% sequence identity to SEQ ID NO: 56; and a CDR3 amino acid sequence having at least about 80% sequence identity to a sequence selected from the group consisting of SEQ ID NOs: 57 and 117.

The antibody or antibody fragment may include a CD4 amino acid sequence having at least about 80% sequence identity to SEQ ID NOs: 58.

The antibody or antibody fragment may include a VHH antibody sequence having at least about 80% sequence identity with a sequence selected from the group consisting of SEQ ID NOs: 25-27.

In some embodiments, the antibody or antibody fragment described herein binds to more than one PFAS compound selected from the group consisting of PFNA, PFOS, PFOA, PFBS, PFHxS, and GenX.

In some embodiments, the antibody or antibody fragment which binds to more than one PFAS compound has an amino acid sequence selected from the group consisting of SEQ ID NOs: 1, 3, 13-17 and 23.

In some embodiments, the antibody or antibody fragment includes one or more variants of one or more CDRs, wherein the one or more variants comprises one or more replacements of hydrophilic amino acid residues with hydrophobic residues, which exhibits greater binding affinity for a PFAS compound.

In some embodiments, the antibody or antibody fragment includes an affinity tag configured to promote binding of a detection molecule.

According to another embodiment, there is provided an isolated nucleic acid sequence encoding any one of the antibodies or antibody fragments described herein.

In some embodiments, the isolated nucleic acid sequence has a sequence selected from the group consisting of SEQ ID NOs: 28-54.

According to another embodiment, there is provided a cell or virus comprising an isolated nucleic acid sequence described herein.

According to another embodiment, there is provided detector system for identification of a PFAS compound comprising an antibody or antibody fragment described herein.

In some embodiments of the detector system, the PFAS compound binds to the antibody or antibody fragment in an ELISA assay.

According to another aspect, there is provided an antibody or antibody fragment which binds at least to perfluorooctanoic acid (PFOA), which includes a CDR1 amino acid sequence having at least about 80% sequence identity to SEQ ID NO: 139; a CDR2 amino acid sequence having at least about 80% sequence identity to SEQ ID NO: 140; and a CDR3 amino acid sequence having at least about 80% sequence identity to SEQ ID NO: 141. This antibody or antibody fragment may include a CD4 amino acid sequence having at least about 80% sequence identity to a sequence selected from the group consisting of SEQ ID NO: 142. This antibody or antibody fragment may have a VHH antibody sequence having at least about 80% sequence identity with SEQ ID NO: 125. In some embodiments, there is provided an isolated nucleic acid sequence encoding the VHH antibody, which may be the sequence of SEQ ID NO: 129.

According to another aspect, there is provided an antibody or antibody fragment which binds at least to perfluorooctanoic acid (PFOA), which includes a CDR1 amino acid sequence having at least about 80% sequence identity to SEQ ID NO: 93; a CDR2 amino acid sequence having at least about 80% sequence identity to SEQ ID NO: 94; and a CDR3 amino acid sequence having at least about 80% sequence identity to SEQ ID NO: 95. This antibody or antibody fragment may include a CD4 amino acid sequence having at least about 80% sequence identity to a sequence selected from the group consisting of SEQ ID NO: 58. This antibody or antibody fragment may have a VHH antibody sequence having at least about 80% sequence identity with SEQ ID NO: 17. In some embodiments, there is provided an isolated nucleic acid sequence encoding the VHH antibody, which may be the sequence of SEQ ID NO: 44.

According to another aspect, there is provided an antibody or antibody fragment which binds at least to PFNA which includes a CDR1 amino acid sequence having at least about 80% sequence identity to SEQ ID NO: 55; a CDR2 amino acid sequence having at least about 80% sequence identity to SEQ ID NO: 56; and a CDR3 amino acid sequence having at least about 80% sequence identity to SEQ ID NO: 57. This antibody or antibody fragment may include a CD4 amino acid sequence having at least about 80% sequence identity to a sequence selected from the group consisting of SEQ ID NO: 59. This antibody or antibody fragment may have a VHH antibody sequence having at least about 80% sequence identity with SEQ ID NO: 2. In some embodiments, there is provided an isolated nucleic acid sequence encoding the VHH antibody, which may be the sequence of SEQ ID NO: 29.

According to another aspect, there is provided an antibody or antibody fragment which binds at least to perfluorooctanoic acid (PFOA), which includes a CDR1 amino acid sequence having at least about 80% sequence identity to SEQ ID NO: 131; a CDR2 amino acid sequence having at least about 80% sequence identity to SEQ ID NO: 132; and a CDR3 amino acid sequence having at least about 80% sequence identity to SEQ ID NO: 133. This antibody or antibody fragment may include a CD4 amino acid sequence having at least about 80% sequence identity to a sequence selected from the group consisting of SEQ ID NO: 134. This antibody or antibody fragment may have a VHH antibody sequence having at least about 80% sequence identity with SEQ ID NO: 123. In some embodiments, there is provided an isolated nucleic acid sequence encoding the VHH antibody, which may be the sequence of SEQ ID NO: 127.

According to another aspect, there is provided an antibody or antibody fragment which binds at least to perfluorooctanoic acid (PFOA), which includes a CDR1 amino acid sequence having at least about 80% sequence identity to SEQ ID NO: 135; a CDR2 amino acid sequence having at least about 80% sequence identity to SEQ ID NO: 136; and a CDR3 amino acid sequence having at least about 80% sequence identity to SEQ ID NO: 137. This antibody or antibody fragment may include a CD4 amino acid sequence having at least about 80% sequence identity to a sequence selected from the group consisting of SEQ ID NO: 138. This antibody or antibody fragment may have a VHH antibody sequence having at least about 80% sequence identity with SEQ ID NO: 124. In some embodiments, there is provided an isolated nucleic acid sequence encoding the VHH antibody, which may be the sequence of SEQ ID NO: 128.

According to another aspect, there is provided an antibody or antibody fragment which binds at least to perfluorooctanoic acid (PFOA), which includes a CDR1 amino acid sequence having at least about 80% sequence identity to SEQ ID NO: 143; a CDR2 amino acid sequence having at least about 80% sequence identity to SEQ ID NO: 144; and a CDR3 amino acid sequence having at least about 80% sequence identity to SEQ ID NO: 145. This antibody or antibody fragment may include a CD4 amino acid sequence having at least about 80% sequence identity to a sequence selected from the group consisting of SEQ ID NO: 59. This antibody or antibody fragment may have a VHH antibody sequence having at least about 80% sequence identity with SEQ ID NO: 126. In some embodiments, there is provided an isolated nucleic acid sequence encoding the VHH antibody, which may be the sequence of SEQ ID NO: 130.

In some embodiments, there is provided a cell or virus including any of the isolated nucleic acid sequences described herein.

In some embodiments, the antibody or antibody fragment described herein binds to one or more additional PFAS compounds selected from the group consisting of PFNA, PFOS, PFBS, PFHxS, and GenX.

According to another aspect, there is provided a detector system for identification of a PFAS compound, which includes any one or more of the antibodies or antibody fragments described herein. In some embodiments of the detector system, the PFAS compound binds to the antibody or antibody fragment in an ELISA assay. The ELISA assay may be a direct ELISA or a competitive ELISA.

In some embodiments, the ELISA assay is configured to detect free PFAS compounds in an aqueous sample. The free PFAS compounds may comprise one or more PFAS compounds selected from the group consisting of: PFOA, PFNA, PFOS, PFBS, PFHxS, and GenX.

In some embodiments, the ELISA assay comprises an immobilized PFAS compound. The immobilized PFAS compound may be immobilized using a biotin-streptavidin interaction. The competitive ELISA may include binding of a PFAS molecule conjugated to a biotin moiety.

In some embodiments, the detector system include an assay plate coated with streptavidin, wherein the PFAS molecule conjugated to the biotin moiety provides the immobilized PFAS compound upon binding of the biotin moiety to the streptavidin.

In some embodiments, the VHH antibody includes an affinity tag and a secondary antibody binds to the affinity tag.

In some embodiments, the competitive ELISA includes a tertiary antibody conjugated to a peroxidase enzyme, wherein the tertiary antibody binds to the secondary antibody, thereby providing a detectable antibody complex via a peroxidase substrate reaction.

In some embodiments, the competitive ELISA includes a cyclodextrin which binds to the free PFAS compound and to the immobilized PFAS compound.

### BRIEF DESCRIPTION OF THE DRAWINGS

Various objects, features and advantages of particular embodiments of the disclosure will be apparent from the following description and the accompanying figures.

FIG. 1A is a gel RNA profile obtained from lymphocytes.

FIG. 1B is a gel VHH DNA profile following nested PCR showing bands from 600 bp to 900 bp.

FIG. 1C is a gel VHH DNA profile following nested PCR showing bands from 400 bp and below.

FIG. 2 is a gel VHH profile for 19 randomly picked colonies of transformants of bacterial cells with VHH vectors for a phage display library.

FIG. 3A is a bar chart indicating ELISA detection of binding of VHH antibody of PFNA V2 to PFNA in a methanol solution in carbonate buffer with control samples.

FIG. 3B is a bar chart indicating ELISA detection of binding of VHH antibody of PFNA V2 to PFNA in a DMSO solution in carbonate buffer with control samples.

FIG. 4 is a plot of absorbance vs. concentration (ppm) of free PFAS in solution as measured in a competitive ELISA assay with PFNA coated plates.

FIG. 5 is a plot of absorbance vs. concentration (ppm) of free PFAS in solution as measured in a direct ELISA assay with a PFAS compound immobilized on plates using a streptavidin-biotin interaction.

FIG. 6 is a bar chart showing results of a direct ELISA assay analysis of VHH antibodies PFNA V1 to PFNA V8, with absorbance measured in the absence of immobilized PFAS compound and in the presence of 200 ppm of immobilized PFAS compound.

FIG. 7 is a bar chart showing results of a direct ELISA assay analysis of VHH antibodies PFOA V1 to PFOA V6, PFBS V1 to PFBS V3, PFOS V1 and PFOS v2, with absorbance measured in the absence of immobilized PFAS compound and in the presence of 200 ppm of immobilized PFAS compound.

FIG. 8 is a bar chart showing results of a direct ELISA assay analysis of VHH antibodies PFHxS V1 to PFHxS V6, and GenX V1 to GenX V3, with absorbance measured in the absence of immobilized PFAS compound and in the presence of 200 ppm of immobilized PFAS compound.

FIG. 9 is a bar chart showing results of a direct ELISA assay analysis of VHH antibodies BPL1 to BPL4, with absorbance measured in the absence of immobilized PFAS compound and in the presence of 200 ppm of immobilized PFAS compound.

FIG. 10 is a bar chart comparing the results of the direct ELISA assays of FIGS. 6 to 9, for the VHH antibodies displaying the greatest extent of binding to the immobilized PFAS compound.

FIG. 11 is a bar chart comparing the results of the direct ELISA assays of the three VHH antibodies (PFNA V2, PFOA V5 and BPL3) displaying the greatest extent of binding to the immobilized PFAS compound.

FIG. 12 is a plot of absorbance vs. concentration of free PFOA (ppm) for the three VHH antibodies (PFNA V2, PFOA V5 and BPL3) displaying the greatest extent of binding to the immobilized PFAS compound. EC10 and ECos values calculated from these plots are indicated for each VHH antibody.

### DETAILED DESCRIPTION

### Introduction

The present inventors have been engaged in development of field deployable electrochemical detectors for analyzing environmental samples for various analytes (see for example, U.S. Pat. Nos. 9,689,046 and 10,415,102, which are each incorporated herein by reference in entirety).

In recognizing a need for rapid detection of PFAS compounds in environmental samples, the inventors recognized that antibodies could be generated to selectively bind to PFAS molecules and used as the basis for a biosensor-based initial detection step in combination with a detector molecule recognizing a binding complex between an antibody and a given PFAS compound, wherein the recognition of the binding complex leads to release of an electrochemically or spectrophotometrically detectable molecule whose signal is correlated with the level of PFAS molecule in an environmental sample. The inventors selected the VHH antibody class as the basis for the antibodies because this class of antibodies was expected to provide superior binding results for binding PFAS molecules relative to the larger antibodies such as immunoglobulins (IgGs).

### Antibodies

As used herein, an “antibody” generally refers to a protein consisting of one or more polypeptides substantially encoded by immunoglobulin genes or fragments of immunoglobulin genes. Where the term “antibody” is used, the term “antibody fragment” may also be considered to be referred to. The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. The basic immunoglobulin (antibody) structural unit is known to comprise a tetramer or dimer. Each tetramer is composed of two identical pairs of polypeptide chains, each pair having one “light” (L) (about 25 kD) and one “heavy” (H) chain (about 50-70 kD). The N-terminus of each chain defines a variable region of about 100 to 110 or more amino acids, primarily responsible for antigen recognition. The terms “variable light chain” and “variable heavy chain” refer to these variable regions of the light and heavy chains respectively. Optionally, the antibody or the immunological portion of the antibody, can be chemically conjugated to, or expressed as, a fusion protein with other proteins. The antibodies described herein are intended to bind to PFAS compounds.

“Specific binding” is to be understood as via one skilled in the art, whereby the skilled person is clearly aware of various experimental procedures that can be used to test binding and binding specificity. Some cross-reaction or background binding may be inevitable in many protein-protein interactions; this is not to detract from the “specificity” of the binding between antibody and epitope. The term “directed against” is also applicable when considering the term “specificity” in understanding the interaction between antibody and epitope.

Embodiments of antibodies include, but are not limited to polyclonal, monoclonal, bispecific, human, humanized or chimeric antibodies, single variable fragments (ssFv), single domain antibodies (such as VHH fragments from nanobodies), single chain fragments (scFv), Fab fragments, F(ab′)2 fragments, fragments produced by a Fab expression library, anti-idiotypic antibodies and epitope-binding fragments or combinations thereof of any of the above, provided that they retain the original binding properties provided by the CDR sequences or variants thereof. Also mini-antibodies and multivalent antibodies such as diabodies, triabodies, tetravalent antibodies and peptabodies can be generated using the antibodies described herein. Examples of such multivalent antibodies are expected to have enhanced binding affinity for PFAS compounds. The immunoglobulin molecules can be of any class (i.e. IgG, IgE, IgM, IgD and IgA) or subclass of immunoglobulin molecules. Thus, the term antibody, as used herein, also includes antibodies and antibody fragments either produced by the modification of whole antibodies or synthesized de novo using recombinant DNA methodologies.

The present invention further relates to the use of the antibodies, or fragments thereof, as described herein, for example the variable regions, in recognition molecules or affinity reagents that are suitable for selective binding to a target. The antibody or fragment thereof according to the invention may be chemically modified by covalent attachment of chemical or biochemical affinity moieties to the antibody using conventional conjugation techniques to provide flexibility in development of various detection assays, including but not limited to ELISA assays, which may be competitive assays, non-competitive assays, direct assays, indirect assays, or sandwich-type ELISA assays.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) which are also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability; and (2) an approach based on crystallographic studies of antigen-antibody complexes. As used herein, a CDR may refer to CDRs defined by either approach or by a combination of both approaches.

In some embodiments, the invention provides an antibody, which comprises at least one CDR, at least two, at least three, or more CDRs that are substantially identical to at least one CDR, at least two, at least three, or more CDRs of the antibody of the invention. Other embodiments include antibodies which have at least two, three, four, five, or six CDR(s) that are substantially identical to at least two, three, four, five or six CDRs of the antibodies of the invention or derived from the antibodies of the invention. In some embodiments, the at least one, two, three, or four CDR(s) are at least about 75%, 76%, 77%, 78%, 79%, 80%, 81%, 82%, 83%, 84%, 85%, 86%, 87%, 88%, 89%, 90%, 95%, 96%, 97%, 98%, or 99% identical to at least one, two, or three CDRs of the antibody of the invention. It is understood that, for purposes of this invention, binding specificity and/or overall activity is generally retained, although the extent of activity may vary compared to said antibody (may be greater or lesser). Using conventional genetic engineering techniques, the CDRs of the VHH antibodies described herein may be provided on alternative antibody frameworks which may be chimeric antibodies of various classes and formed of domains from different species. Such modifications may be provided for any reason, such as for example, providing increased stability in detection systems.

Sequence variants of the claimed nucleic acids, proteins and antibodies, for example defined by the claimed % sequence identity, that maintain the said properties of the invention are also included in the scope of the invention. Such variants, which show alternative sequences, but maintain essentially the same binding properties, such as target specificity, as the specific sequences provided are known as functional analogues, or as functionally analogous. Sequence identity relates to the percentage of identical nucleotides or amino acids when carrying out a sequence alignment.

It will be appreciated by those of ordinary skill in the art that, as a result of the degeneracy of the genetic code, there are many nucleotide sequences that encode a polypeptide as described herein. Some of these polynucleotides bear minimal homology or sequence identity to the nucleotide sequence of any native gene. Nonetheless, polynucleotides that vary due to differences in codon usage are specifically contemplated by the present invention. Deletions, substitutions and other changes in sequence that fall under the described sequence identity are also encompassed in the invention.

Protein sequence modifications, which may occur through substitutions, are also included within the scope of the invention. Substitutions as defined herein are modifications made to the amino acid sequence of the protein, whereby one or more amino acids are replaced with the same number of (different) amino acids, producing a protein which contains a different amino acid sequence than the primary protein, preferably without significantly altering the function of the protein. Additions and/or substitutions may be natural or artificial. It is well known in the art that amino acid substitutions may be made without significantly altering the protein's function. This is particularly true when the modification relates to a “conservative” amino acid substitution, which is the substitution of one amino acid for another of similar properties. Such “conserved” amino acids can be natural or synthetic amino acids which because of size, charge, polarity and conformation can be substituted without significantly affecting the structure and function of the protein. Frequently, many amino acids may be substituted by conservative amino acids without deleteriously affecting the protein's function.

In general, the non-polar amino acids Gly, Ala, Val, Ile and Leu; the non-polar aromatic amino acids Phe, Trp and Tyr; the neutral polar amino acids Ser, Thr, Cys, Gln, Asn and Met; the positively charged amino acids Lys, Arg and His; the negatively charged amino acids Asp and Glu, represent groups of conservative amino acids. This list is not exhaustive. For example, it is well known that Ala, Gly, Ser and sometimes Cys can substitute for each other even though they belong to different groups.

Substitution variants have at least one amino acid residue in the antibody molecule removed and a different residue inserted in its place. The sites of greatest interest for substitutional mutagenesis include the hypervariable regions, but FR alterations are also contemplated.

According to some embodiments, one or more hydrophilic amino acid residues of one or more CDRs of the inventive antibodies are replaced with one or more hydrophobic amino acid residues with the aim of enhancing binding affinity for PFAS compounds through hydrophobic interactions.

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain.

Conservative amino acid substitutions are not limited to naturally occurring amino acids, but also include synthetic amino acids. Commonly used synthetic amino acids are omega amino acids of various chain lengths and cyclohexyl alanine which are neutral non-polar analogs; citrulline and methionine sulfoxide which are neutral non-polar analogs, phenylglycine which is an aromatic neutral analog; cysteic acid which is a negatively charged analog and ornithine which is a positively charged amino acid analog. Like the naturally occurring amino acids, this list is not exhaustive, but merely exemplary of the substitutions that are well known in the art.

The antibodies of the present invention may be produced by transfection of a host cell with an expression vector comprising the coding sequence for the antibody of the invention. An expression vector or recombinant plasmid is produced by placing these coding sequences for the antibody in operative association with conventional regulatory control sequences capable of controlling the replication and expression in, and/or secretion from, a host cell. Regulatory sequences include promoter sequences, e.g., CMV or T7 promoters, and signal sequences which can be derived from other known antibodies. Similarly, a second expression vector can be produced having a DNA sequence which encodes a complementary antibody light or heavy chain. In certain embodiments, this second expression vector is identical to the first except insofar as the coding sequences and selectable markers are concerned, so to ensure as far as possible that each polypeptide chain is functionally expressed. Alternatively, the heavy and light chain coding sequences for the antibody may reside on a single vector. Antibodies described herein are VHH antibodies. However, the CDRs of the antibodies described herein may be engineered into any of the classes of immunoglobulins, such as IgG, IgM, IgA, IgD and IgE as well as any antibody fragments thereof, to provide chimeric antibodies or fragments thereof.

A selected host cell is co-transfected by conventional techniques with both the first and second vectors (or simply transfected by a single vector) to create the transfected host cell of the invention comprising both the recombinant or synthetic light and heavy chains. The transfected cell is then cultured by conventional techniques to produce the engineered antibody of the invention. The antibody which includes the association of both the recombinant heavy chain and/or light chain is screened from culture by appropriate assay, such as ELISA or RIA. Similar conventional techniques may be employed to construct other antibodies.

Suitable vectors for the cloning and subcloning steps employed in the methods and construction of the compositions of this invention may be selected by one of skill in the art. For example, the conventional pUC series of cloning vectors may be used. In one example, the vector known as pUC19, is commercially available. The components of such vectors, e.g. replicons, selection genes, enhancers, promoters, signal sequences and the like, may be obtained from commercial or natural sources or synthesized by known procedures for use in directing the expression and/or secretion of the product of the recombinant DNA in a selected host. Other appropriate expression vectors of which numerous types are known in the art for mammalian, bacterial, insect, yeast, and fungal expression may also be selected for this purpose.

The present invention also encompasses a cell line transfected with a recombinant plasmid containing the coding sequences of the antibodies of the present invention. Host cells useful for the cloning and other manipulations of these cloning vectors are also conventional.

Suitable host cells or cell lines for the expression of the antibodies of the invention include mammalian cells such as NS0, Sp2/0, CHO (e.g. DG44), COS, HEK, a fibroblast cell (e.g., 3T3), and myeloma cells, for example it may be expressed in a CHO or a myeloma cell. Human cells may be used, thus enabling the molecule to be modified with human glycosylation patterns. Alternatively, other prokaryotic or eukaryotic cell lines may be employed such as E. coli, P. pastoris, or S. cerevisiae. The selection of suitable mammalian or prokaryotic host cells and methods for transformation, culture, amplification, screening and product production and purification are known in the art.

### PFAS Compounds

The United States Environmental Protection Agency (EPA) curates a master list of PFAS compounds on its internet site currently at comptox.epa.gov. Some of the more common PFAS compounds include perfluorononanoic acid (PFNA), perfluorooctanoic acid (PFOA), perfluorooctane sulfonate (PFOS), perfluorohexane sulfonate (PFHxS), perfluorobutane sulfonate (PFBS), and hexafluoropropylene oxide dimer acid (HFPO-DA, also commonly known as GenX). Notably, each of these compounds includes at least one ionizable functional group which will be protonated or deprotonated, depending on the pH of the solution and the pKa value of each individual ionizable group. For the sake of simplicity, the compound names listed above are intended herein to refer to both the protonated and deprotonated forms as well as salts thereof.

In considering the challenges associated with raising antibodies that bind to small molecules, the present inventors recognized that VHH antibodies are well suited for small molecule analysis. A VHH antibody (also known as a “nanobody”) is the antigen binding fragment of antibodies which consist of only a heavy chain and are naturally produced by camelids and sharks. Discovered nearly 25 years ago, VHH antibodies have been investigated for their use in clinical therapeutics and immunodiagnostics, and more recently for environmental monitoring applications. A new and valuable immunoreagent for the analysis of small molecular weight environmental chemicals, VHH antibodies are expected to overcome many pitfalls encountered with conventional reagents. In the work so far, VHH antibodies often perform comparably to conventional antibodies for small molecule analysis, are amenable to numerous genetic engineering techniques, and show ease of adaption to other immunodiagnostic platforms for use in environmental monitoring (Bever et al., Anal. Bioanal. Chem., 2016, 408 (22): 5985-6002, incorporated herein by reference in its entirety).

In a typical conventional process for isolation of VHH antibodies from camelids which bind to a given compound, the camelids are injected with a solution containing the compound and blood is collected from the camelids, from which mRNA is collected and converted into cDNA by reverse transcriptase PCR. The cDNA is amplified and digested to isolate the VHH genes, for incorporation into plasmids and expressed by a bacteriophage, thereby creating a VHH library. The library is then panned to identify the VHH antibodies of interest (Bever et al., infra). Following isolation of VHH antibodies, testing is conducted to determine binding affinity for binding of the VHH antibodies to the compound of interest.

The present application describes antibodies and fragments thereof having binding affinity for PFAS compounds, including, but not limited to six of the most common and potentially dangerous PFAS compounds; PFNA, PFOA, PFOS, PFHxS, PFBS, and GenX.

The invention includes antibodies which bind to more than one of the compounds listed above, including two or more, three or more, four or more, five or more, or all six of PFNA, PFOA, PFOS, PFHxS, PFBS, and GenX. Some antibodies may also bind additional PFAS compounds including, but not limited to PFAS compounds listed in the master list of PFAS compounds curated by the EPA at comptox.epa.gov.

### Detection of PFAS Compounds

The antibodies described herein were developed to provide the basis for a biosensor for detecting PFAS compounds. In one embodiment, the biosensor includes an ELISA-based recognition assay where specific binding of an antibody to a given PFAS compound generates an immobilized complex which is detected by a recognition molecule such as a secondary antibody or affinity-based molecule conjugated to an enzyme. The enzyme catalyzes a reaction of a precursor molecule to generate a detectable molecule which is quantifiable by electrochemical, spectrophotometric, colorimetric or fluorometric techniques. In one example of an ELISA-based biosensor assay, an environmental sample is provided for testing of the presence of a PFAS compounds. An anti-PFAS antibody recognizing a PFAS compound is contacted with the sample and incubated under conditions where the resulting complex is immobilized on a substrate. The sample is washed, and a secondary antibody developed to recognize the anti-PFAS compound antibody is added to the sample. The secondary antibody includes conjugated horseradish peroxidase. A peroxidase substrate such as 3,3′,5,5′-tetramethylbenzidine (TMB) is added to the sample and is converted by the peroxidase to an electrochemically active species which provides an electrochemical signal correlated with the level of PFNA in the sample. Examples of enzymes and molecules providing electrochemically active molecules in a field deployable chemical detector are described in U.S. Pat. Nos. 9,689,046 and 10,415,102, which are each incorporated herein by reference in entirety.

In some detector embodiments, a PFAS compound is immobilized on a substrate and pre-mixed with the antibody or antibody fragment. The antibody will bind to any PFAS compound in the sample, meaning there are less available antibodies or antibody fragments to interact with the PFAS compound on the substrate. The decrease in signal observed will be proportional to the concentration of PFAS compound in the original sample. This can be done at the same time as exposure to the plate or in the pre-mixed sample. Alternatively, a PFAS compound could be immobilized on the plate as “free” PFAS, or through conjugation, e.g., labeling BSA with the PFAS compound, or using a streptavidin-coated plate and exposing it to a biotin-conjugated PFAS compound.

Another detector embodiment could use a capture antibody on the surface such as an anti-camelid or anti-hemagglutinin antibody and exposing it to an anti-PFAS antibody or antibody fragment at the same time or in a separate tube containing the sample of interest with a known concentration of a “detectable” PFAS compound. This detectable compound could be radio labelled, conjugated to an enzyme, or conjugated to another moiety that can later be detected, for example a conjugated biotin to provide an interaction with streptavidin-linked peroxidase, exposed subsequently. Other interacting moieties may be used instead of biotin and streptavidin in alternative embodiments. The greater the concentration of PFAS compound in the sample, the smaller the fraction of detectable PFAS compound will be bound by the antibody or antibody fragment, which would translate to less detectable PFAS captured on the surface.

In some embodiments, the antibody backbone may be tuned to decrease overall size, to add additional recognition sites for secondary antibodies (e.g., to add a GST tag), to add cleavage tags (e.g., TEV before the purification tag), or to directly attach HRP/AP/LacZ/enzymatic reporters such that a secondary antibody is not needed. The antibody may include an affinity tag such as polyhistidine, maltose binding protein, glutathione-S-transferase, Strep-tag, calmodulin binding protein, FLAG tag, S-tag, chitin-binding domain, hemagglutinin, and c-Myc tag. These and other affinity tags and tagging strategies are described in Zhao et al., J. Anal, Methods Chem., 2013, Article ID 581093, incorporated herein by reference in its entirety). The antibody may include a non-enzymatic reporter molecule such as green fluorescent protein, luciferase and anthocyanin, for example.

In some embodiments, instead of a secondary antibody, the peroxidase enzyme is linked to an affinity tag such as a small polypeptide, a small molecule or a metal chelate and the anti-PFAS antibody is provided with a moiety for that binds to the affinity tag, if required. In other embodiments, an affinity tag may be provided on the antibody or antibody fragment and a binding sequence may be provided on a reporter molecule.

In certain preferred embodiments of the inventive biosensor system, the mixing, reactions and detection occur in channels and chambers of a cartridge and the reagents outlined above are provided together with the cartridge in a kit product. The kit may include instructions for carrying out the sample assay. Some embodiments of the kit may include an antibody which can bind to more than one PFAS compound for use as a general survey assay while other embodiments may include a series of antibodies for specific detection of specific PFAS compounds such as any one of the PFAS compounds identified herein, as well as the PFAS compounds of the master list curated by the United States EPA, for example.

### EXAMPLES

### Example 1: Generation of VHH Antibodies Recognizing PFAS Compounds

Preparation of Antigen Solutions—Antigen solutions of PFAS compounds were prepared according to the protocols provided by the commercial suppliers of the PFAS compounds. PFNA (CAS #335-95-1) was obtained from Fischer Scientific (Hampton, NH, USA), PFOA (CAS #335-67-1) was obtained from Sigma-Aldrich (St. Louis, MO, USA), PFOS (CAS #1763-23-1) was obtained from Sigma-Aldrich, PFHxS (CAS #355-46-4) was obtained from Clinisciences (Nanterre, France), PFBS (CAS #375-73-5) was obtained from Sigma Aldrich, and GenX (CAS #13252-13-6) was obtained from Wellington Laboratories (Guelph, ON, Canada).

Immunization—Healthy 2-year old dromedary camels (Camelus dromedarius) were immunized primarily with PFAS compound samples mixed with an equal volume of Freund's complete and incomplete adjuvant. To stimulate antigen-specific B cells, the PFAS compound samples were injected every week. After eight injections and 3-4 days after the last injection, 100 mL of anticoagulated blood was collected from the jugular vein of the immunized animal. Using a Leucosep® tube, the blood was concentrated to a final volume of 30 mL. After centrifugation at room temperature for 10 min at 1,000 xg, the plasma (top layer) was removed without disturbing the interface and the peripheral blood lymphocytes (PBLs) were collected at the interphase. PBLs were then isolated by Ficoll-Paque™ PLUS (GE Healthcare, USA) following the manufacturer's instructions and used to construct the library.

ELISA—To evaluate specificity of ELISA, at a 1:2000 dilution, serum was adsorbed on 96-well Maxisorp™ microtiter plates and incubated overnight at 4° C. The following day, the solution was removed the wells were rinsed 5 times with PBS/Tween. Then 200 μL of 5% (w/v) skimmed milk powder in PBS was added to each antigen-coated well for 2 h at room temperature to block residual protein-binding sites on the plastic. After removing the skimmed milk solution from the wells, 100 μL of anti-IgG antibody conjugated to horseradish peroxidase diluted 1/3,000 in PBS was added the solution was left to stand at room temperature for 1 h. The wells were rinsed 5 times with PBS/Tween, the absorbance was measured at 405 nm after 10-30 min with a microtiter plate reader after adding the ELISA substrate.

Construction of the VHH Library—Total RNA was isolated from peripheral blood lymphocytes using the mammalian Total RNA Miniprep Kit (Sigma-Aldrich). To avoid contamination with VH genes, the variable regions of heavy-chain immunoglobulins (VHH) were amplified by nested PCR. First-strand cDNA was used in each PCR reaction with primers CALL001 (GTCCTGGCTGCTCTTCTACAAGG, SEQ ID NO: 118) and CALL002 (GGTACGTGCTGTTGAACTGTTCC, SEQ ID NO: 119). The protocol of the first PCR consisted of an initial denaturation step at 94° C. for 7 min, followed by 30 cycles of 94° C. for 1 min, 55° C. for 1 min, and 72° C. for 1 min, and a final extension step at 72° C. for 10 min. The VHH genes of the first PCR products were analyzed by agarose gel electrophoresis. Gel plugs from the bands near 700 base pairs (bp) were used to extract DNA, used as template in the secondary PCR. This was performed with the primers VHH BACK-1 (GATGTGCAGCTGCAGGAGTCTGGAGGAGG, SEQ ID NO: 120), VHH-BACK-2 (GATGTGCAGCTGCAGGAGTCTGGGGGAGG, SEQ ID NO: 121), and PMCF (CTAGTGCGGCCGCTGAGGAGACGGTGACCTGGGT, SEQ ID NO: 122) containing restriction enzyme sites PstI and NotI, using the same protocol as the first PCR. The PCR products were run on 1% agarose gel, and the band at approximately 400 bp was extracted from the gel.

The amplified second PCR products (1 μg) were digested with PstI and NotI restriction enzymes (NEB, France), then inserted into a linearized phagemid (3 μg). Ligation products were transformed into Escherichia coli TG1 cells by electroporation. The transformants were plated onto 2×YT containing 10% glucose and 100 μg/mL ampicillin and cultured at 37° C. for 16 h. Plating an aliquot of the library and counting the colony number determined the library size. Many clones were selected randomly and used in a colony PCR to estimate the percentage of clones with a proper insert size within the library. The initial denaturation step was done at 95° C. for 5 min to lyse bacterial cells, followed by 28 cycles of 94° C. for 45 s, 55° C. for 45 s, and 72° C. for 45 s, and a final extension step at 72° C. for 10 min.

Selection of VHH Antibodies-1 mL of the VHH library was cultured and infected with M13 helper phages to express the VHH at the tip of phage particles. PFAS compound samples as antigen (10 μg) in coating buffer (0.1 M NaHCO₃, pH 8.2) were coated onto microtiter plates (Nunc Immuno Maxsorp, Roskilde, Denmark) at 4° C. overnight. The control was 0.1 M NaHCO₃(pH 8.2). After blocking with 2% milk in phosphate-buffered saline (PBS) for 2 h and incubation with phage-displayed sdAbs in PBS for 1 h at room temperature, the specific phages were eluted with 100 mM triethylamine, transferred to a fresh tube, immediately neutralized with 1.0 M Tris-HCl (pH 7.4) and used to infect TG1 cells. This process represented one round of panning. Then, a portion of the TG1 cell sample was plated at various dilutions (ranging from 10-1 to 10-7) whereas the remaining of the culture was super-infected with helper phages M13. The generated phage particles were used in the next round of panning. During two to three rounds of panning, protein-specific phages were enriched gradually.

Periplasmic Extract ELISA—To detect antigens-specific clones, 192 clones were selected randomly for periplasmic extract ELISA. After disrupting the cells by osmotic shock and a centrifugation step to provide the periplasmic extract, the VHH antibodies resided in the supernatant, which was incubated with antigen coated in microtiter plates. Each clone was cultured in 1 mL LB medium with 100 μg/mL ampicillin, then the expression of VHH by 1 mM isopropyl β-d-1-thiogalactopyranoside (IPTG) was induced. Cells were collected and resuspended into 200 μL TES (0.5 M sucrose, 0.2 M Tris-HCl pH 8.0, 0.5 mM EDTA) for 30 min at 4° C., and 300 μL cold TES/4 was added for 30 minutes. The supernatant was transferred into the wells of the microtiter plates with coated antigen (1 μg/mL). After 1 h, mouse anti-HIS tag antibody diluted 1/1,000 in PBS (Thermo Fisher Scientific, USA) was added and the sample was left to stand for 1 h. Then anti-mouse IgG-HRP (Sigma-Aldrich, Saint Louis, USA) diluted 1/2,000 in PBS and the sample was left to stand for 1 h. After washing with PBST (PBS with 0.05% Tween 20) and addition of the chromogenic solution ABTS (Sigma-Aldrich, Saint Louis, MO, USA), the absorbance was measured at 405 nm with an ELISA reader.

Results of Immunization—To verify an immune response, serum was analyzed before injection, after 28 days and then three days after the last injection. The results are shown in Table 1 and indicate an immune response generated for each one of the PFAS compounds.

TABLE 1
Analysis of Serum for Immune Response

| | | | Absorbance |
|---|---|---|---|
| | Absorbance | Absorbance | 3 Days After Last |
| PFAS Compounds | Day 0 | Day 28 | Injection |
| PFOA | 0.105 | 0.7 | 1.36 |
| PFOS | 0.122 | 1.55 | 1.63 |
| PFNA | 0.13 | 0.52 | 2.3 |
| PFBS | 0.22 | 1.18 | 1.98 |
| PFHxS | 0.14 | 1.079 | 1.96 |
| GenX | 0.25 | 1.42 | 1.32 |

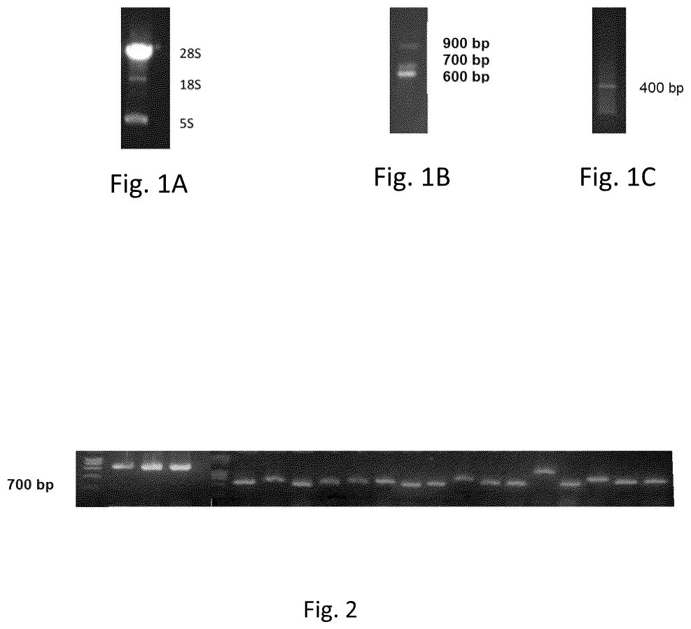

Results-Construction of Immunized Phage Display Library—The VHH sequences were amplified from lymphocyte cDNA of the camels. First, RNA was extracted from lymphocytes obtained from the collected blood (FIG. 1A). Two PCR products including a 900 bp fragment for VH-CH1-CH2 exons and 600 bp for VHH-CH2 exons (FIG. 1B) were amplified with primers CALL001 and CALL002. The gene for the VHH domain of about 400 bp (FIG. 1C) was amplified with nested primers PMCF, BACK-1, and BACK-2 using the 600 bp PCR fragment as a template.

For library construction, two restriction enzymes, PstI and NotI, were introduced into the 5′ and 3′ ends of the final VHH PCR fragments, respectively. In total, 1 μg of purified VHH PCR product and 3 μg of linearized vector were used for the ligation. A total of 25 electroporations were performed to transform the ligation mixture into bacterial cells and to obtain a high-quality library with great diversity. The size of the constructed library was calculated from the number of independent colonies on plates and shown to reach 7×108 colonies. Colony PCR analysis on 19 randomly picked colonies revealed that plasmids having an insert of VHH reached 95% (FIG. 2). Sequencing of these clones indicated that the VHH fragments have unique sequences. These results demonstrate successful construction of a phage display library.

The VHH library generated from PFOA included 1.5×107 clones. The VHH library generated from PFOS included 1.0×107 clones. The VHH library generated from PFNA included 1.2×104 clones. The VHH library generated from PFBS included 1.5×107 clones. The VHH library generated from PFHxS included 5.0×106 clones. The VHH library generated from GenX included 2.0×106 clones.

Bio-panning was performed to isolate VHHs against inactivated proteins. A total of approximately 2×1011 phage particles from the library were used in panning. After three rounds of biopanning, 192 colonies were randomly picked and tested to determine whether the ELISA would reveal the presence of VHHs binding to the PFAS compounds.

Results of ELISA—the ELISA results identified groups of VHH antibodies which bind to the PFAS compounds. The amino acid sequences of the VHH antibodies are shown in Tables 2 to 7, where VHH antibody name designations and sequence identifiers are listed. DNA sequences encoding the VHH antibodies are shown in Tables 8 to 13. CDR sequences of the VHH antibodies are listed in Tables 14 to 19.

TABLE 2
Amino Acid Sequences of Antibodies Generated Against PFNA

| Antibody | | |
|---|---|---|
| Name | Amino Acid Sequence | SEQ ID NO: |
| PFNA V1 | DVQLQESGGGSVQMGGSLRLSCAASEFTWE | 1 |
| | TDQMAWFRQAPGQEREPVARIGGGVLQLRTY | |
| | ADYVKGRFTISRDNAKNMVYLQMNSLKPEDTA | |
| | IYYCAAGPSSTWDIGAYRYWGQGTQVTVSS | |
| PFNA V2 | MGGSLRLSCAASEFTWETDQMAWFRQAPGQ | 2 |
| | EREPVARIGGGVLQLRTYADYVKGRFTISRDN | |
| | AKNTVYLQMNSLKPEDTAIYYCAAGPSSTWDI | |
| | GAYRYWGQGTQVTVSS | |
| PFNA V3 | DVQLQESGGGLVQPGGSLRLSCAASEFTWET | 3 |
| | DQMAWFRQAPGQEREPVARIGGGVLQLRTYA | |
| | DYVKGRFTISRDNAKNMVYLQMNSLKPEDTAI | |
| | YYCAAGPSSTWDIGAYRYWGQGTQVTVSS | |
| PFNA V4 | DVQLQESGGGLVQPGGSLRLSCAASEFTWET | 4 |
| | DQMAWFCQAPGQEREPVARIGGGVLQLRTYA | |
| | DYVKGRFTISRDNAKNMVYLQMNSLKPEDTAI | |
| | YYCAAGPSSTWDIGAYRYWGQGTQVTVSS | |
| PFNA V5 | DVQLQESGGGSVQAGGSLRLSCTVSGYTYSS | 5 |
| | CSMGWYRQAPGKKRELVSSILTNGTTDYADF | |
| | VKGRFTISQDNAQNTVYLQMNSLTPEDTAMYY | |
| | CNNRRAADCSRGGPQYGFMYWGQGTQVTVS | |
| | S | |
| PFNA V6 | DVQLQESGGGLVQPGGSLRLSCAASEFTWET | 6 |
| | DQMAWFRQAPGQEREPVARIGGGVLQLRTYA | |
| | DYVKGRFTISRDNAKNMVYLQMNSLKPGDTAI | |
| | YYCAAGPSSTWDIGAYRYWGQGTQVTVSS | |
| PFNA V7 | DVQLQESGGGLVQPGGSLRLSCAASGFTFNT | 7 |
| | AKMGWFRQTQGKDCELVSTTWPDGRTNVAD | |
| | SVKGRFTISRDNAKNTLYLQLNSLETEDTAMY | |
| | YCTLGQWDYGARGYQGQGTQVTVSS | |
| PFNA V8 | DVQLQESGGGSVQAGGSLKLSCAASGFMSSN | 8 |
| | CMGWFRQAPGKEREGVAVINGVGTTYYANSV | |
| | KGRFTISRDVAKAKNTIYLQMNSLKSEDTATYY | |
| | CAADPAGGVCDLSMSFNSWGQGTQVTVSS | |

TABLE 3
Amino Acid Sequences of Antibodies Generated Against PFOS

| Antibody | | |
|---|---|---|
| Name | Amino Acid Sequence | SEQ ID NO: |
| PFOS V1 | DVQLQESGGGSVQAGGSLRLSCKASGYIYSSY | 9 |
| | CMGWFRQAPGKEREGVAAIHSGGGSTVFLDSV | |
| | RGRFTISRDNARNTVYLQMNSLKPDDTAMYYC | |
| | ATGRAVVPACGERRVGYNYWGQGTQVTVSS | |
| PFOS V2 | DVQLQESGGGSVQAGESLRLTCIIAGCTYCKFE | 10 |
| | MTWYRQTPTNEREFVAGFDSDGGERYGDSVR | |
| | GRSSISRDTTKDKFFLEMNNLKPEDAGMYFCKM | |
| | GSGFCVVTGRDQPEYWGQGTQVTVSS | |

TABLE 4
Amino Acid Sequences of Antibodies Generated Against PFBS

| Antibody | | |
|---|---|---|
| Name | Amino Acid Sequence | SEQ ID NO: |
| PFBS V1 | DVQLQESGGGSVQMGGSLRLSCAASEFTWET | 11 |
| | DQMAWFRQAPGQEREPVARIGGGALQLRTYAD | |
| | YVKGRFTISRDNAKNMVYLQMNSLKPEDTAIYY | |
| | CAAGPSSTWDIGAYRYWGQGTQVTVSS | |
| PFBS V3 | DVQLQESGGGSVQAGGSLRLSCVFSTYRYCTY | 12 |
| | DMTWYRQAPGKEREFVSIIDRAGSTTYADSVKG | |
| | RFTISQDNAKNTVYLQMNSLKPEDTAIYYCAARP | |
| | PVLGIYCHGEVAMWNYWGQGTQVTVSS | |
| PFBS V2 | DVQLQESGGGSVQMGGSLRLSCAASEFTWET | 1 |
| | DQMAWFRQAPGQEREPVARIGGGVLQLRTYAD | |
| | YVKGRFTISRDNAKNMVYLQMNSLKPEDTAIYY | |
| | CAAGPSSTWDIGAYRYWGQGTQVTVSS | |

TABLE 5
Amino Acid Sequences of Antibodies Generated Against PFOA

| Antibody | | |
|---|---|---|
| Name | Amino Acid Sequence | SEQ ID NO: |
| PFOA V2 | DVQLQESGGGSVQTGGSLRLSCTAPGFTANMC | 14 |
| | GMAYFRQTGGNPRVWVASIGTYGDIKYADPVK | |
| | GRFTISRDSAKDTVYLQMNNLQPEDTGLYTCKA | |
| | NPGVADKCWGYNYWSQGTQVTVSS | |
| PFOA V1 | DVQLQESGGGSVQAGGSLRLSCVASDYTYDFY | 15 |
| | CMGWFRQVPGKEREGVAVTNKAGVTTFYGDS | |
| | VKNRFTISQADVENTVYLEMNGLKPEDTAIYYCV | |
| | LRSSAACPTGSWSSSLNYHTWGRGTQVTVSS | |
| PFOA V3 | DVQLQESGGGSVPAGGSLRLSCAASGYTFSSC | 16 |
| | CMAWFRQAPGKEREGVASINSGGGTTSYADSV | |
| | KGRFTISRDNAKNTTYLQMNSLKPEDTAMYYCA | |
| | AEGPVCGGTYPSLFRGVYNTWGQGTQVTVSS | |
| PFOA V5 | DVQLQESGGGSVQTGGSLRLSCAASGFTFSSH | 17 |
| | WMYWVRQAPGKGLEWVSTVNWGGGSTYYAD | |
| | SVKGRFTISRDNAKNMLYLQMNSLKPEDTALYY | |
| | CGEGLDFGYWGQGTQVTVSS | |
| PFOA V6 | DVQLQESGGGLVQPGGSLRLSCAATEFTWETD | 18 |
| | QMAWFRQAPGQEREPVARIGGGVLQLRTYADY | |
| | VKGRFTISRDNAKNMVYLQMNSLKPEDTAIYYC | |
| | AAGPSSTWDIGAYRYWGQGTQVTVSS | |

TABLE 6
Amino Acid Sequences of Antibodies Generated Against PFHxS

| Antibody | | |
|---|---|---|
| Name | Amino Acid Sequence | SEQ ID NO: |
| PFHxS V1 | DVQLQESGGGSVQAGESLRLSCAASGITSGAN | 19 |
| | YMGWFRQAPGKERDWVAVIHFGSGTTYYADSV | |
| | AGRFTFSQDNAKDTFYLQMDSLKPEVSASYYC | |
| | ATGLVGGGTWSCGYNYWGQGTQVTVSS | |
| PFHxS V2 | DVQLQESGGGSVQAGGSLRLSCAASGDRNCK | 20 |
| | YDMSWYRQAPGKEREFVSVIDNDGRSRYADSV | |
| | KGRFTISQDNAKSTVYLQMNSLKPEDTAMYYCA | |
| | AGYPDYSLLLGTPGYDFWGQGTQVTVSS | |
| PFHxS V3 | DVQLQESGGGSVQAGGSLRLSCVASGHFHRTY | 21 |
| | CMGWFRQAPGKEREGVASIFSDGSSTYYADSV | |
| | KGRFTISQDNAKNTVYLQMNTLQPEDTAIYVCA | |
| | AETRIDLCNEGSFFTTARYRGQGTQVTVSS | |
| PFHxS V4 | DVQLQESGGGLVQPGGSLRLSCAASGFTFTNY | 22 |
| | YMYWLRQAPGKGLEWVSTINFDGSNTGYANSV | |
| | KGRFTISRDNAKNTLYLQMNSLKSEDTALYYCA | |
| | TDDVGGERGQGTQVTVSS | |
| PFHxS V5 | DVQLQESGGGSVQTGESLRLSCTVSGYTNTKD | 23 |
| | YIGWFRQAPGKEREGLALFFTDDGTAWFTDSV | |
| | KDRFALAQENTKNTVSLQMNRLKPSDTAMYFC | |
| | ATNTVGSWSWPLVPTDFHFWGQGTQVTVSS | |
| PFHxS V6 | DVQLQESGGGLVQPAGSLRLSCVASGFTFDNY | 24 |
| | AMGWLRQAPGKEIEWVATINSEGKTSYADSVQ | |
| | DRFIISRDNARNTLYLELNSLKIEDTAKYYCVRG | |
| | RSPSVLVTTDPPSGQGTQVTVSS | |

TABLE 7
Amino Acid Sequences of Antibodies Generated Against GenX

| Antibody | | |
|---|---|---|
| Name | Amino Acid Sequence | SEQ ID NO: |
| GenX V1 | DVQLQESGGGLVQPGGSLRLSCAASEFTWETD | 25 |
| | QMAWFRQAPGQEREPVARIGGGVLQLRTYADY | |
| | VKGRFTISRDNAKNMVYLQMNSLKPEDTAIYYC | |
| | AAGPSSTWDIGAYRHWGQGTQVTVSS | |
| GenX V3 | DVQLQESGGGSVQMGGSLRLSCAASEFTWET | 26 |
| | DQMAWFRQAPGQEREPVARIGGGVLQLRTYAD | |
| | YVKGRFTISRDNAKNMVYLQMNSLKPEDTAIYY | |
| | CAAGPSSTWDIGAYRYWGQGTQVTVSSAAAD | |
| | GSSPVSWRATSGYKYWGQGTQVTVSS | |
| GenX V2 | DVQLQESGGGLVQPGGSLRLSCAASEFTWETD | 27 |
| | QMAWSRQAPGQEREPVARIGGGVLQLRTYADY | |
| | VKGRFTISRDNAKNMVYLQMNSLKPEDTAIYYC | |
| | AAGPSSTWDIGAYRYWGQGTQVTVSS | |

TABLE 8
DNA Sequences Encoding Antibodies Generated
Against PFNA

| Anti- | | SEQ |
|---|---|---|
| body | | ID |
| Name | DNA Sequence | NO: |
| PFNA V1 | GACGTCCAGCTGCAGGAATCTGGCGGTGGCT | 28 |
| | CCGTGCAGATGGGTGGCTCCCTGCGTCTGTCT | |
| | TGTGCAGCGTCCGAATTCACCTGGGAAACTGA | |
| | TCAGATGGCGTGGTTCCGTCAGGCGCCGGGC | |
| | CAGGAACGTGAACCGGTAGCTCGTATTGGCG | |
| | GTGGCGTTCTGCAGCTGCGCACTTATGCAGAC | |
| | TATGTTAAAGGCCGTTTTACGATTAGCCGCGAT | |
| | AACGCCAAGAACATGGTTTACCTGCAGATGAA | |
| | CAGCCTGAAGCCGGAGGATACTGCTATTTATT | |
| | ACTGCGCAGCCGGTCCGTCTTCCACTTGGGAC | |
| | ATCGGCGCTTATCGTTACTGGGGCCAGGGCA | |
| | CTCAGGTGACGGTGAGCTCC | |
| PFNA V2 | ATGGGTGGCAGCCTGCGTCTGAGCTGCGCGG | 29 |
| | CATCTGAGTTTACGTGGGAAACTGACCAGATG | |
| | GCCTGGTTCCGTCAGGCGCCGGGTCAGGAAC | |
| | GCGAACCGGTGGCGCGTATCGGTGGCGGTGT | |
| | TCTGCAGCTGCGTACGTACGCGGACTACGTAA | |
| | AAGGCCGTTTCACCATCTCCCGTGACAACGCT | |
| | AAAAATACCGTATATCTGCAGATGAACTCTCTG | |
| | AAACCGGAAGATACCGCTATCTACTATTGTGCT | |
| | GCCGGTCCGAGCTCCACTTGGGACATTGGCG | |
| | CCTATCGTTATTGGGGTCAAGGTACCCAGGTA | |
| | ACCGTTTCCAGC | |
| PFNA V3 | GACGTCCAGCTGCAGGAATCCGGTGGCGGTC | 30 |
| | TGGTTCAACCGGGTGGCTCTCTGCGCCTGTCC | |
| | TGTGCGGCTAGCGAATTCACCTGGGAAACTGA | |
| | TCAGATGGCATGGTTTCGCCAGGCTCCGGGTC | |
| | AGGAACGTGAACCTGTAGCACGCATCGGTGG | |
| | CGGTGTCCTGCAGCTGCGTACCTACGCCGATT | |
| | ACGTTAAAGGCCGCTTCACCATCTCTCGTGAC | |
| | AACGCGAAAAACATGGTATACCTGCAAATGAA | |
| | CAGCCTGAAGCCGGAAGATACGGCCATCTATT | |
| | ACTGTGCGGCTGGCCCGAGCTCCACTTGGGA | |
| | TATTGGCGCGTACCGTTACTGGGGTCAGGGTA | |
| | CCCAAGTAACGGTAAGCTCT | |
| PFNA V4 | GACGTCCAACTGCAGGAAAGCGGTGGCGGTC | 31 |
| | TGGTTCAGCCAGGTGGCTCCCTGCGTCTCAGC | |
| | TGCGCGGCCTCTGAATTCACTTGGGAAACCGA | |
| | TCAGATGGCCTGGTTCTGCCAGGCGCCAGGT | |
| | CAGGAGCGTGAGCCGGTTGCTCGCATCGGCG | |
| | GTGGCGTACTGCAGCTGCGTACTTACGCGGAT | |
| | TATGTGAAAGGTCGCTTTACTATCTCTCGCGAT | |
| | AACGCTAAAAATATGGTGTACCTGCAGATGAA | |
| | CTCCCTGAAGCCGGAAGACACGGCTATCTATT | |
| | ACTGCGCAGCCGGTCCGTCCAGCACTTGGGA | |
| | CATCGGTGCTTATCGTTACTGGGGCCAGGGCA | |
| | CTCAGGTGACTGTCAGCAGC | |
| PFNA V5 | GACGTACAGCTGCAGGAAAGCGGCGGTGGCA | 32 |
| | GCGTTCAAGCTGGCGGTTCTCTGCGCCTGTCT | |
| | TGCACCGTGTCCGGCTACACCTACTCCTCTTG | |
| | CTCTATGGGCTGGTACCGCCAGGCACCGGGT | |
| | AAAAAGCGTGAACTGGTTTCTAGCATTCTGAC | |
| | CAACGGTACTACCGATTATGCCGACTTCGTAA | |
| | AAGGCCGTTTTACCATCTCTCAGGACAACGCG | |
| | CAGAATACCGTGTACCTGCAGATGAACAGCCT | |
| | GACCCCGGAAGATACGGCAATGTATTACTGCA | |
| | ATAACCGTCGCGCAGCTGACTGTTCCCGTGGC | |
| | GGTCCACAGTACGGCTTTATGTATTGGGGCCA | |
| | GGGCACGCAGGTAACGGTTTCTAGC | |
| PFNA V6 | GATGTCCAGCTGCAGGAAAGCGGCGGTGGCC | 33 |
| | TGGTTCAGCCGGGCGGTTCCCTGCGCCTGAG | |
| | CTGCGCTGCGTCTGAATTCACCTGGGAAACCG | |
| | ATCAAATGGCGTGGTTCCGCCAGGCCCCAGG | |
| | TCAAGAGCGTGAACCGGTGGCGCGTATCGGC | |
| | GGTGGCGTACTGCAACTGCGTACCTACGCGG | |
| | ATTACGTTAAAGGCCGTTTCACCATTAGCCGT | |
| | GATAACGCTAAAAACATGGTTTACCTGCAGAT | |
| | GAACTCCCTGAAGCCTGGCGACACCGCTATTT | |
| | ATTACTGCGCGGCCGGTCCGTCCAGCACTTG | |
| | GGATATTGGCGCTTACCGCTACTGGGGTCAGG | |
| | GTACGCAGGTTACCGTATCCTCC | |
| PFNA V7 | GACGTTCAACTCCAAGAATCTGGTGGCGGTCT | 34 |
| | GGTGCAGCCGGGCGGTTCTCTGCGTCTGTCC | |
| | TGTGCGGCATCCGGCTTTACCTTCAACACTGC | |
| | TAAGATGGGTTGGTTCCGCCAGACGCAGGGC | |
| | AAAGATTGTGAACTGGTCAGCACGACCTGGCC | |
| | GGATGGTCGTACTAATGTGGCTGATTCCGTAA | |
| | AAGGCCGCTTTACTATCTCTCGCGACAACGCG | |
| | AAGAACACCCTGTATCTGCAGCTGAACTCCCT | |
| | GGAAACTGAGGACACCGCAATGTATTACTGTA | |
| | CCCTGGGTCAGTGGGATTATGGTGCGCGTGG | |
| | CTATCAGGGTCAGGGCACCCAAGTTACCGTGT | |
| | CCTCT | |
| PFNA V8 | GATGTACAGCTGCAGGAGTCTGGCGGTGGCA | 35 |
| | GCGTACAGGCGGGCGGTTCTCTGAAACTGTCT | |
| | TGCGCAGCCAGCGGCTTCATGTCTAGCAACTG | |
| | CATGGGTTGGTTTCGTCAGGCACCGGGTAAAG | |
| | AACGTGAAGGCGTAGCTGTTATCAACGGTGTT | |
| | GGCACTACGTACTATGCGAACTCTGTAAAAGG | |
| | TCGTTTTACGATTTCCCGTGATGTTGCGAAAGC | |
| | GAAGAACACCATCTATCTGCAAATGAACTCTCT | |
| | GAAAAGCGAAGATACCGCTACCTACTATTGCG | |
| | CTGCCGATCCGGCGGGCGGTGTCTGCGACCT | |
| | GTCTATGTCTTTCAACAGCTGGGGTCAGGGTA | |
| | CCCAGGTTACGGTTAGCAGC | |

TABLE 9
DNA Sequences Encoding Antibodies Generated
Against PFOS

| | | SEQ |
|---|---|---|
| Antibody | | ID |
| Name | DNA Sequence | NO: |
| PFOS V1 | GACGTACAGCTGCAGGAATCTGGTGGCGGT | 36 |
| | TCTGTCCAAGCGGGTGGCTCTCTGCGCCTG | |
| | AGCTGCAAAGCGAGCGGTTACATTTACTCCT | |
| | CTTATTGTATGGGCTGGTTCCGTCAAGCGCC | |
| | GGGTAAAGAACGTGAAGGTGTTGCCGCGAT | |
| | TCATAGCGGCGGTGGCTCCACTGTATTCCTC | |
| | GATTCTGTACGTGGTCGTTTCACTATTAGCC | |
| | GTGACAACGCCCGTAACACCGTCTATCTGCA | |
| | GATGAACTCTCTGAAACCGGATGACACTGC | |
| | GATGTATTACTGCGCGACCGGTCGCGCTGT | |
| | AGTTCCTGCTTGTGGTGAGCGTCGCGTTGG | |
| | CTATAACTATTGGGGCCAGGGTACTCAGGTT | |
| | ACCGTTTCTAGC | |
| PFOS V2 | GACGTCCAGCTGCAGGAATCTGGTGGCGGT | 37 |
| | TCCGTTCAGGCAGGTGAGTCCCTGCGTCTG | |
| | ACTTGCATTATCGCTGGCTGCACTTACTGCA | |
| | AATTTGAAATGACCTGGTACCGTCAGACTCC | |
| | GACCAACGAACGCGAATTTGTTGCGGGTTTC | |
| | GATAGCGACGGTGGCGAACGTTACGGTGAT | |
| | TCTGTTCGTGGTCGCTCCTCTATTAGCCGTG | |
| | ATACTACCAAGGACAAGTTCTTTCTGGAAAT | |
| | GAATAACCTGAAACCGGAAGATGCTGGCAT | |
| | GTACTTCTGCAAAATGGGCTCCGGCTTCTGC | |
| | GTGGTCACTGGCCGTGACCAGCCGGAATAT | |
| | TGGGGTCAGGGCACGCAGGTAACGGTTTCC | |
| | TCT | |

TABLE 10
DNA Sequences Encoding Antibodies Generated
Against PFBS

| | | SEQ |
|---|---|---|
| Antibody | | ID |
| Name | DNA Sequence | NO: |
| PFBS V1 | GATGTCCAGCTGCAAGAGTCCGGTGGCGGT | 38 |
| | TCCGTTCAAATGGGCGGTTCCCTGCGCCTG | |
| | TCTTGTGCTGCATCCGAGTTCACTTGGGAAA | |
| | CCGATCAGATGGCGTGGTTCCGTCAGGCTC | |
| | CGGGTCAGGAACGTGAGCCGGTTGCTCGTA | |
| | TTGGCGGTGGCGCTCTGCAGCTGCGTACCT | |
| | ATGCAGACTATGTGAAAGGTCGCTTTACCAT | |
| | TAGCCGCGACAACGCTAAAAACATGGTGTA | |
| | CCTGCAGATGAACTCCCTGAAACCGGAAGA | |
| | TACCGCCATTTATTACTGCGCGGCTGGTCCA | |
| | TCTTCCACGTGGGACATTGGCGCCTACCGT | |
| | TACTGGGGTCAGGGCACCCAGGTCACTGTT | |
| | TCTTCT | |
| PFBSV3 | GACGTCCAGCTCCAGGAATCTGGTGGCGGT | 39 |
| | TCTGTTCAGGCGGGTGGCTCCCTGCGCCTG | |
| | TCTTGCGTTTTCAGCACCTATCGTTACTGTA | |
| | CTTACGACATGACCTGGTATCGCCAGGCGC | |
| | CGGGTAAGGAACGTGAGTTCGTTAGCATTAT | |
| | CGACCGCGCTGGCTCCACTACCTACGCTGA | |
| | CTCCGTAAAAGGTCGTTTCACCATCTCTCAG | |
| | GACAACGCCAAAAACACTGTCTATCTGCAGA | |
| | TGAACTCTCTGAAGCCGGAGGATACGGCTA | |
| | TTTATTACTGTGCTGCGCGTCCGCCTGTTCT | |
| | GGGTATTTACTGCCATGGTGAAGTGGCAAT | |
| | GTGGAACTACTGGGGTCAGGGCACCCAGGT | |
| | GACCGTTTCCTCT | |
| PFBS V2 | GATGTACAGCTGCAAGAAAGCGGTGGCGGT | 40 |
| | TCCGTCCAGATGGGTGGCTCTCTGCGCCTC | |
| | TCTTGCGCTGCATCCGAATTCACCTGGGAG | |
| | ACTGATCAAATGGCATGGTTCCGTCAAGCG | |
| | CCGGGTCAAGAACGTGAGCCGGTTGCACGC | |
| | ATCGGTGGCGGTGTGCTGCAGCTGCGTACC | |
| | TACGCAGACTACGTGAAAGGTCGCTTCACC | |
| | ATTTCTCGTGACAACGCTAAAAACATGGTTT | |
| | ACCTGCAAATGAACTCTCTCAAACCTGAGGA | |
| | TACCGCGATCTATTACTGTGCGGCTGGTCC | |
| | GAGCTCCACGTGGGACATTGGTGCTTATCG | |
| | TTACTGGGGTCAGGGTACGCAGGTGACCGT | |
| | GAGCAGC | |

TABLE 11
DNA Sequences Encoding Antibodies Generated
Against PFOA

| | | SEQ |
|---|---|---|
| Antibody | | ID |
| Name | DNA Sequence | NO: |
| PFOA V2 | GACGTTCAACTGCAAGAATCTGGTGGCGGT | 41 |
| | AGCGTCCAAACCGGCGGTTCTCTCCGCCTG | |
| | TCTTGCACTGCGCCTGGTTTCACCGCCAACA | |
| | TGTGCGGCATGGCCTATTTCCGTCAGACTG | |
| | GTGGCAACCCACGCGTTTGGGTAGCCTCCA | |
| | TTGGTACCTACGGCGACATTAAATACGCGGA | |
| | CCCGGTGAAGGGCCGTTTCACCATCTCTCG | |
| | CGATTCTGCGAAAGACACTGTGTACCTGCA | |
| | GATGAATAACCTGCAACCGGAAGACACCGG | |
| | CCTGTACACCTGCAAAGCTAACCCGGGCGT | |
| | AGCAGACAAATGCTGGGGTTACAACTATTGG | |
| | TCCCAGGGTACCCAAGTTACTGTGTCCTCT | |
| PFOA V1 | GATGTTCAGCTGCAGGAGTCCGGCGGTGGC | 42 |
| | AGCGTACAAGCAGGCGGTTCCCTGCGCCTG | |
| | TCCTGTGTCGCTTCTGACTACACCTACGATT | |
| | TTTACTGCATGGGTTGGTTCCGCCAGGTTCC | |
| | GGGCAAAGAACGTGAAGGTGTGGCCGTTAC | |
| | CAACAAAGCAGGTGTTACGACCTTCTACGGT | |
| | GACTCTGTTAAAAACCGCTTCACCATCAGCC | |
| | AAGCTGACGTCGAGAATACTGTTTACCTGGA | |
| | AATGAACGGCCTGAAACCGGAAGACACTGC | |
| | GATCTACTATTGCGTGCTGCGTTCCAGCGCA | |
| | GCGTGCCCGACCGGCTCCTGGTCCAGCTCC | |
| | CTCAATTACCATACCTGGGGTCGCGGCACC | |
| | CAAGTTACCGTCAGCTCC | |
| PFOA V3 | GACGTGCAACTGCAGGAATCTGGTGGCGGT | 43 |
| | TCTGTTCCGGCTGGCGGTTCTCTGCGTCTG | |
| | AGCTGCGCTGCCTCCGGCTATACCTTTTCTA | |
| | GCTGTTGCATGGCTTGGTTTCGCCAGGCGC | |
| | CGGGCAAAGAACGTGAAGGCGTCGCGAGC | |
| | ATCAACAGCGGCGGTGGCACTACCTCCTAC | |
| | GCTGATTCTGTTAAGGGCCGTTTCACCATCT | |
| | CTCGTGACAACGCTAAAAACACGACCTACCT | |
| | GCAGATGAATAGCCTGAAACCGGAAGACAC | |
| | TGCAATGTACTATTGCGCAGCGGAAGGTCC | |
| | GGTATGCGGCGGTACGTATCCGAGCCTGTT | |
| | TCGTGGTGTATATAACACCTGGGGCCAGGG | |
| | CACCCAGGTGACGGTTTCTTCT | |
| PFOA V5 | GACGTTCAGCTGCAGGAATCTGGTGGCGGT | 44 |
| | TCTGTTCAGACCGGTGGCTCTCTGCGCCTG | |
| | TCTTGCGCTGCGAGCGGCTTCACCTTTAGCT | |
| | CTCACTGGATGTACTGGGTTCGTCAGGCCC | |
| | CGGGTAAAGGCCTGGAATGGGTGTCCACGG | |
| | TTAACTGGGGTGGCGGTTCCACTTACTATGC | |
| | AGATTCTGTGAAAGGCCGCTTCACCATTTCC | |
| | CGTGATAATGCAAAAAACATGCTGTACCTGC | |
| | AGATGAATAGCCTGAAACCGGAAGATACCG | |
| | CTCTGTATTACTGCGGTGAAGGCCTGGACTT | |
| | CGGTTACTGGGGCCAGGGCACTCAGGTGAC | |
| | CGTGTCTTCT | |
| PFOA V6 | GACGTACAACTGCAGGAATCTGGCGGTGGC | 45 |
| | CTGGTCCAGCCTGGCGGTTCCCTGCGTCTC | |
| | AGCTGCGCTGCGACCGAGTTCACGTGGGAA | |
| | ACCGATCAGATGGCTTGGTTCCGTCAGGCT | |
| | CCGGGTCAGGAACGTGAACCGGTGGCGCG | |
| | CATCGGCGGTGGCGTACTGCAGCTCCGTAC | |
| | CTACGCGGACTACGTTAAGGGTCGCTTCAC | |
| | CATCTCTCGTGACAACGCGAAAAACATGGTG | |
| | TATCTGCAGATGAATTCTCTGAAACCGGAAG | |
| | ACACCGCTATCTATTACTGCGCTGCAGGCC | |
| | CGTCCTCTACCTGGGACATCGGCGCGTACC | |
| | GTTATTGGGGCCAGGGCACCCAGGTTACCG | |
| | TATCCAGC | |

TABLE 12
DNA Sequences Encoding Antibodies Generated
Against PFHxS

| | | SEQ |
|---|---|---|
| Antibody | | ID |
| Name | DNA Sequence | NO: |
| PFHxS V1 | GACGTACAGCTGCAAGAGAGCGGCGGTGG | 46 |
| | CAGCGTGCAGGCGGGTGAGTCCCTGCGTCT | |
| | GTCTTGCGCGGCCTCTGGTATTACCTCTGG | |
| | CGCTAACTACATGGGTTGGTTTCGCCAGGC | |
| | TCCGGGTAAAGAACGTGACTGGGTAGCGGT | |
| | TATCCATTTTGGCTCTGGTACTACCTATTAC | |
| | GCTGACAGCGTAGCTGGTCGTTTCACTTTTT | |
| | CTCAAGACAACGCTAAAGACACTTTCTACCT | |
| | GCAGATGGACTCCCTGAAACCGGAAGTTTC | |
| | TGCGTCCTACTATTGCGCGACCGGCCTGGT | |
| | GGGTGGCGGTACTTGGAGCTGCGGTTATAA | |
| | CTACTGGGGTCAGGGCACTCAGGTAACCGT | |
| | CTCTTCT | |
| PFHxS V2 | GACGTCCAGCTGCAGGAATCCGGCGGTGG | 47 |
| | CTCCGTACAGGCGGGTGGCAGCCTGCGCCT | |
| | GTCTTGCGCTGCGTCTGGTGACCGTAACTG | |
| | TAAATACGATATGTCTTGGTACCGTCAAGCG | |
| | CCAGGCAAGGAACGCGAATTCGTCTCCGTT | |
| | ATCGACAACGATGGCCGTTCCCGTTATGCA | |
| | GACTCCGTTAAAGGCCGTTTCACCATCTCTC | |
| | AAGATAACGCGAAATCTACCGTATACCTGCA | |
| | AATGAATTCTCTCAAACCGGAAGATACTGCC | |
| | ATGTACTATTGCGCGGCTGGTTATCCGGATT | |
| | ATTCTCTGCTCCTGGGTACGCCAGGCTATGA | |
| | TTTCTGGGGCCAGGGTACCCAAGTTACGGT | |
| | TTCCTCT | |
| PFHxS V3 | GATGTGCAGCTGCAGGAGTCTGGCGGTGGC | 48 |
| | TCTGTTCAGGCGGGTGGCTCTCTGCGTCTG | |
| | TCTTGTGTTGCGAGCGGTCACTTCCACCGTA | |
| | CCTACTGCATGGGTTGGTTCCGTCAAGCGC | |
| | CGGGCAAGGAACGTGAAGGTGTGGCGAGC | |
| | ATCTTCTCTGACGGCTCTAGCACGTACTATG | |
| | CGGACAGCGTTAAAGGCCGTTTCACCATCA | |
| | GCCAAGATAACGCAAAAAACACCGTTTATCT | |
| | GCAGATGAACACCCTGCAGCCGGAAGACAC | |
| | TGCTATCTACGTTTGCGCTGCAGAAACCCGT | |
| | ATCGACCTGTGCAATGAAGGCTCTTTCTTTA | |
| | CTACCGCACGTTATCGTGGCCAGGGCACGC | |
| | AAGTTACCGTTTCCTCC | |
| PFHxS V4 | GACGTTCAGCTGCAGGAAAGCGGCGGTGG | 49 |
| | CCTGGTCCAGCCAGGTGGCTCTCTGCGTCT | |
| | GTCCTGCGCAGCCTCCGGTTTTACCTTCACC | |
| | AACTACTATATGTACTGGCTGCGTCAGGCTC | |
| | CGGGTAAAGGCCTGGAATGGGTCTCTACTA | |
| | TCAACTTCGATGGCAGCAACACTGGTTACGC | |
| | GAACTCTGTAAAGGGTCGCTTCACGATCAG | |
| | CCGTGACAATGCGAAAAACACCCTGTACCT | |
| | GCAGATGAACTCTCTCAAGTCCGAAGACAC | |
| | CGCGCTGTATTACTGTGCTACCGACGATGTA | |
| | GGCGGTGAACGTGGTCAGGGCACCCAAGT | |
| | GACCGTTTCTTCC | |
| PFHxS V5 | GATGTTCAACTGCAGGAAAGCGGCGGTGGC | 50 |
| | TCCGTACAGACCGGCGAAAGCCTGCGCCTG | |
| | AGCTGCACCGTATCCGGCTACACCAACACC | |
| | AAAGATTACATCGGCTGGTTCCGTCAGGCTC | |
| | CTGGTAAAGAACGTGAAGGCCTGGCTCTGT | |
| | TCTTTACTGACGATGGTACCGCCTGGTTCAC | |
| | CGACAGCGTTAAAGATCGCTTCGCGCTGGC | |
| | TCAGGAAAACACTAAAAACACCGTGAGCCTG | |
| | CAGATGAACCGTCTGAAGCCTTCCGACACG | |
| | GCCATGTATTTTTGTGCTACCAACACCGTTG | |
| | GTTCTTGGTCCTGGCCTCTGGTTCCGACCG | |
| | ATTTCCATTTTTGGGGCCAGGGTACTCAGGT | |
| | TACCGTTTCTAGC | |
| PFHxS V6 | GACGTACAGCTGCAGGAGTCCGGTGGCGGT | 51 |
| | CTGGTTCAGCCGGCGGGTTCTCTGCGCCTG | |
| | TCTTGCGTTGCATCCGGCTTCACCTTTGATA | |
| | ACTACGCTATGGGTTGGCTGCGCCAGGCGC | |
| | CGGGTAAAGAAATTGAATGGGTTGCGACCA | |
| | TCAATTCCGAAGGCAAAACCAGCTATGCAGA | |
| | CTCCGTTCAGGACCGCTTCATTATCTCCCGT | |
| | GACAACGCGCGTAATACTCTGTACCTGGAA | |
| | CTGAACAGCCTGAAAATTGAAGATACCGCGA | |
| | AATACTATTGCGTTCGTGGCCGCTCTCCGTC | |
| | TGTCCTGGTCACGACTGATCCTCCGAGCGG | |
| | CCAAGGTACGCAAGTCACCGTCTCTAGC | |

TABLE 13
DNA Sequences Encoding Antibodies Generated
Against GenX

| | | SEQ |
|---|---|---|
| Antibody | | ID |
| Name | DNA Sequence | NO: |
| GenX V1 | GACGTTCAGCTGCAGGAGTCTGGTGGC | 52 |
| | GGTCTGGTCCAGCCGGGTGGCAGCCT | |
| | GCGCCTGAGCTGTGCGGCATCTGAGTT | |
| | TACCTGGGAAACCGACCAAATGGCATG | |
| | GTTTCGTCAGGCGCCGGGTCAGGAACG | |
| | TGAGCCGGTCGCACGTATTGGCGGTGG | |
| | CGTACTGCAGCTGCGTACTTATGCAGA | |
| | CTACGTCAAAGGCCGTTTCACCATCAGC | |
| | CGTGACAACGCGAAGAACATGGTTTATC | |
| | TGCAGATGAACTCTCTCAAACCGGAAGA | |
| | TACCGCGATCTACTATTGCGCGGCTGG | |
| | CCCGAGCTCTACTTGGGATATCGGTGC | |
| | GTACCGCCACTGGGGCCAGGGTACGCA | |
| | GGTCACCGTGAGCTCT | |
| GenX V3 | GACGTACAGCTGCAGGAGTCCGGTGGC | 53 |
| | GGTTCCGTGCAGATGGGGGGTTCCCTG | |
| | CGTCTGAGCTGTGCAGCTAGCGAATTC | |
| | ACCTGGGAGACTGATCAGATGGCATGG | |
| | TTCCGTCAGGCGCCGGGCCAAGAACGT | |
| | GAGCCAGTCGCTCGTATCGGTGGCGGT | |
| | GTTCTGCAGCTGCGTACCTACGCAGATT | |
| | ATGTTAAAGGTCGTTTTACGATTTCTCG | |
| | TGACAACGCTAAAAACATGGTTTATCTC | |
| | CAGATGAACAGCCTGAAGCCAGAAGAT | |
| | ACCGCTATCTATTACTGCGCCGCGGGC | |
| | CCTTCCAGCACCTGGGACATCGGTGCG | |
| | TACCGTTACTGGGGCCAGGGTACTCAG | |
| | GTCACTGTTTCCAGCGCGGCCGCTGAT | |
| | GGTTCCAGCCCTGTTTCCTGGCGCGCA | |
| | ACCTCTGGCTACAAATACTGGGGCCAG | |
| | GGTACCCAGGTTACTGTGTCCTCC | |
| GenX V2 | GATGTCCAGCTGCAGGAATCCGGTGGC | 54 |
| | GGTCTGGTTCAGCCGGGCGGTAGCCTG | |
| | CGTCTGAGCTGCGCGGCTTCCGAGTTC | |
| | ACCTGGGAAACCGACCAGATGGCGTGG | |
| | AGCCGTCAGGCGCCGGGCCAGGAACG | |
| | CGAACCGGTCGCGCGTATCGGTGGCG | |
| | GTGTGCTGCAGCTGCGCACTTACGCAG | |
| | ATTACGTGAAAGGTCGTTTCACCATTAG | |
| | CCGCGATAACGCAAAAAACATGGTGTAT | |
| | CTGCAGATGAACTCTCTGAAACCGGAA | |
| | GATACCGCTATCTATTACTGTGCAGCGG | |
| | GCCCATCCTCTACCTGGGACATCGGTG | |
| | CGTACCGTTACTGGGGCCAGGGCACCC | |
| | AGGTCACCGTCTCTTCC | |

TABLE 14
CDR Sequences of Antibodies Generated
Against PFNA

| | | | SEQ |
|---|---|---|---|
| Antibody | | | ID |
| Name | CDR No. | CDR Sequence | NO: |
| PFNA V1 | CDR1 | EFTWETDQMA | 55 |
| PFNA V1 | CDR2 | RIGGGVLQLRT | 56 |
| PFNA V1 | CDR3 | GPSSTWDIGAYRY | 57 |
| PFNA V1 | CDR4 | DNAKNM | 58 |
| PFNA V2 | CDR1 | EFTWETDQMA | 55 |
| PFNA V2 | CDR2 | RIGGGVLQLRT | 56 |
| PFNA V2 | CDR3 | GPSSTWDIGAYRY | 57 |
| PFNA V2 | CDR4 | DNAKNT | 59 |
| PFNA V3 | CDR1 | EFTWETDQMA | 55 |
| PFNA V3 | CDR2 | RIGGGVLQLRT | 56 |
| PFNA V3 | CDR3 | GPSSTWDIGAYRY | 57 |
| PFNA V3 | CDR4 | DNAKNM | 58 |
| PFNA V4 | CDR1 | EFTWETDQMA | 55 |
| PFNA V4 | CDR2 | RIGGGVLQLRT | 56 |
| PFNA V4 | CDR3 | GPSSTWDIGAYRY | 57 |
| PFNA V4 | CDR4 | DNAKNM | 58 |
| PFNA V5 | CDR1 | GYTYSSCSMG | 60 |
| PFNA V5 | CDR2 | SILTNGTTD | 61 |
| PFNA V5 | CDR3 | GGPQYGFMY | 62 |
| PFNA V5 | CDR4 | DNAQNT | 63 |
| PFNA V6 | CDR1 | EFTWETDQMA | 55 |
| PFNA V6 | CDR2 | RIGGGVLQLRT | 56 |
| PFNA V6 | CDR3 | GPSSTWDIGAYRY | 57 |
| PFNA V6 | CDR4 | DNAKNM | 58 |
| PFNA V7 | CDR1 | GFTFNTAKMG | 64 |
| PFNA V7 | CDR2 | TTWPDGRTN | 65 |
| PFNA V7 | CDR3 | GQWDYGARGY | 66 |
| PFNA V7 | CDR4 | DNAKNT | 59 |
| PFNA V8 | CDR1 | GFMSSNCMG | 67 |
| PFNA V8 | CDR2 | VINGVGTTY | 68 |
| PFNA V8 | CDR3 | DPAGGVCDLSMSFNS | 69 |
| PFNA V8 | CDR4 | DVAKAK | 70 |

TABLE 15
CDR Sequences of Antibodies Generated
Against PFOS

| | | | SEQ |
|---|---|---|---|
| Antibody | | | ID |
| Name | CDR No. | CDR Sequence | NO: |
| PFOS V1 | CDR1 | GYIYSSYCMG | 71 |
| PFOS V1 | CDR2 | AIHSGGGSTV | 72 |
| PFOS V1 | CDR3 | RRVGYNY | 73 |
| PFOS V1 | CDR4 | DNARNT | 74 |
| PFOS V2 | CDR1 | GCTYCKFEMT | 75 |
| PFOS V2 | CDR2 | GFDSDGGER | 76 |
| PFOS V2 | CDR3 | TGRDQPEY | 77 |
| PFOS V2 | CDR4 | DAGMYF | 78 |

TABLE 16
CDR Sequences of Antibodies Generated
Against PFBS

| | | | SEQ |
|---|---|---|---|
| Antibody | | | ID |
| Name | CDR No. | CDR Sequence | NO: |
| PFBS V1 | CDR1 | EFTWETDQMA | 55 |
| PFBS V1 | CDR2 | RIGGGALQLRT | 56 |
| PFBS V1 | CDR3 | GPSSTWDIGAYRY | 57 |
| PFBS V1 | CDR4 | DNAKNM | 58 |
| PFBS V3 | CDR1 | TYRYCTYDMT | 79 |
| PFBS V3 | CDR2 | IIDRAGSTT | 80 |
| PFBS V3 | CDR3 | RPPVLGIYCHGEVAMWNY | 81 |
| PFBS V3 | CDR4 | DNAKNT | 59 |
| PFBS V2 | CDR1 | EFTWETDQMA | 55 |
| PFBS V2 | CDR2 | RIGGGVLQLRT | 56 |
| PFBS V2 | CDR3 | GPSSTWDIGAYRY | 57 |
| PFBS V2 | CDR4 | DNAKNM | 58 |

TABLE 17
CDR Sequences of Antibodies Generated
Against PFOA

| | | | SEQ |
|---|---|---|---|
| Antibody | CDR | | ID |
| Name | No. | CDR Sequence | NO: |
| PFOA V2 | CDR1 | GFTANMCGMAYFRQTGGNPRV | 86 |
| PFOA V2 | CDR2 | SRDSAKDT | 87 |
| PFOA V2 | CDR3 | NPGVADKCWGYNY | 88 |
| PFOA V2 | CDR4 | DSAKDT | 89 |
| PFOA V1 | CDR1 | DYTYDFYCMG | 82 |
| PFOA V1 | CDR2 | VTNKAGVTTF | 83 |
| PFOA V1 | CDR3 | GSWSSSLNYHT | 84 |
| PFOA V1 | CDR4 | DTAIYY | 85 |
| PFOA V3 | CDR1 | GYTFSSCCMA | 90 |
| PFOA V3 | CDR2 | SINSGGGTTS | 91 |
| PFOA V3 | CDR3 | TYPSLFRGVYNT | 92 |
| PFOA V3 | CDR4 | DNAKNT | 59 |
| PFOA V5 | CDR1 | GFTFSSHWMY | 93 |
| PFOA V5 | CDR2 | TVNWGGGSTY | 94 |
| PFOA V5 | CDR3 | GLDFGY | 95 |
| PFOA V5 | CDR4 | DNAKNM | 58 |
| PFOA V6 | CDR1 | EFTWETDQMA | 55 |
| PFOA V6 | CDR2 | RIGGGVLQLRT | 56 |
| PFOA V6 | CDR3 | GPSSTWDIGAYRY | 57 |
| PFOA V6 | CDR4 | DNAKNM | 58 |

TABLE 18
CDR Sequences of Antibodies Generated
Against PFHxS

| | | | SEQ |
|---|---|---|---|
| Antibody | | | ID |
| Name | CDR No. | CDR Sequence | NO: |
| PFHxS V1 | CDR1 | GITSGANYMG | 96 |
| PFHxS V1 | CDR2 | VIHFGSGTTY | 97 |
| PFHxS V1 | CDR3 | GLVGGGTWSCGYNY | 98 |
| PFHxS V1 | CDR4 | DNAKDT | 99 |
| PFHxS V2 | CDR1 | GDRNCKYDMS | 100 |
| PFHxS V2 | CDR2 | VIDNDGRSR | 101 |
| PFHxS V2 | CDR3 | GYPDYSLLLGTPGYDF | 102 |
| PFHxS V2 | CDR4 | DNAKST | 103 |
| PFHxS V3 | CDR1 | GHFHRTYCMG | 104 |
| PFHxS V3 | CDR2 | SIFSDGSSTY | 105 |
| PFHxS V3 | CDR3 | ETRIDLCNEGSFFTTARY | 106 |
| PFHxS V3 | CDR4 | DNAKNT | 59 |
| PFHxS V4 | CDR1 | GFTFTNYYMY | 107 |
| PFHxS V4 | CDR2 | TINFDGSNTG | 108 |
| PFHxS V4 | CDR3 | DDVGGE | 109 |
| PFHxS V4 | CDR4 | DNAKNT | 59 |
| PFHxS V5 | CDR1 | GYTNTKDYIG | 110 |
| PFHxS V5 | CDR2 | LFFTDDGTAW | 111 |
| PFHxS V5 | CDR3 | NTVGSWSWPLVPTDFHF | 112 |
| PFHxS V5 | CDR4 | DRFALA | 113 |
| PFHxS V6 | CDR1 | GFTFDNYAMG | 114 |
| PFHxS V6 | CDR2 | TINSEGKTS | 115 |
| PFHxS V6 | CDR3 | GRSPSVLVTTDPP | 116 |
| PFHxS V6 | CDR4 | DNARNT | 74 |

TABLE 19
CDR Sequences of Antibodies Generated
Against GenX

| | | | SEQ |
|---|---|---|---|
| Antibody | | | ID |
| Name | CDR No. | CDR Sequence | NO: |
| GenX V1 | CDR1 | EFTWETDQMA | 55 |
| GenX V1 | CDR2 | RIGGGVLQLRT | 56 |
| GenX V1 | CDR3 | GPSSTWDIGAYRH | 117 |
| GenX V1 | CDR4 | DNAKNM | 58 |
| GenX V3 | CDR1 | EFTWETDQMA | 55 |
| GenX V3 | CDR2 | RIGGGVLQLRT | 56 |
| GenX V3 | CDR3 | GPSSTWDIGAYRY | 57 |
| GenX V3 | CDR4 | DNAKNM | 58 |
| GenX V2 | CDR1 | EFTWETDQMA | 55 |
| GenX V2 | CDR2 | RIGGGVLQLRT | 56 |
| GenX V2 | CDR3 | GPSSTWDIGAYRY | 57 |
| GenX V2 | CDR4 | DNAKNM | 58 |

Among the CDR sequences, the CDR1 sequences have lengths ranging from 9 to 21 amino acid residues, the CDR2 sequences have lengths ranging from 8 to 11 amino acid residues and the CDR3 sequences have lengths ranging from 6 to 18 amino acid residues. Among the CDR sequences, the CDR1 sequence EFTWETDQMA (SEQ ID NO: 55), the CDR2 sequence RIGGGVLQLRT (SEQ ID NO: 56) and the CDR3 sequence GPSSTWDIGAYRY (SEQ ID NO: 57) appear together in 11 different antibodies which were generated against four different PFAS compounds.

Characteristics of the lengths and positions of the three main CDR sequences (CDR1, CDR2 and CDR3) of the group of VHH antibodies of SEQ ID NOs: 1-27 are outlined below. This information was obtained based on analysis workflow developed by Wilton et al. (ACS Synth. Biol. 2018, 7 (11), 2480-2484, incorporated herein by reference in its entirety).

CDR1 has a starting position which includes the first cysteine reside, is followed by three unspecified residues, has a sequence including the cysteine of a length of 6 to 15 residues and is followed by a tryptophan residue. CDR2 begins 10 to 20 residues after the end of CDR1, with a residue selected from isoleucine, leucine, valine and methionine, followed by a residue selected from glycine, alanine and serine, has a length of 8 to 15 residues and is followed by a residue selected from tyrosine, phenylalanine, isoleucine, leucine, threonine, asparagine, serine, valine and histidine, followed by three unspecified residues, followed by a residue selected from alanine, isoleucine, leucine, methionine and valine, followed by a residue selected from glutamine, lysine, arginine, alanine, glutamic acid, glycine, leucine and threonine. CDR3 begins 30 to 50 residues after the end of CDR2, has a sequence beginning with a cysteine residue followed by two unspecified residues, has a length of three to 25 residues and is followed by a residue selected from tryptophan, alanine, glutamic acid, phenylalanine, histidine, lysine, leucine, glutamine, tyrosine, glycine, serine and arginine, followed by a residue selected from glycine and serine, followed by an unspecified residue, followed by glycine, followed by three unspecified residues followed by a residue selected from threonine, valine and serine.

It was determined that among the VHH antibodies with amino acid sequences of SEQ ID NOs: 1-27, certain VHH antibodies bind to more than one of the PFAS compounds. Table 20 lists each of the 27 VHH antibodies and indicates confirmed binding of individual PFAS compounds by each antibody. Notably, antibodies PFNA V1 (SEQ ID NO:1), PFNA V3 (SEQ ID NO: 3), PFBS V2 (SEQ ID NO: 13), PFOA V1 (SEQ ID NO: 15), PFOA V2 (SEQ ID NO: 14), PFOA V3 (SEQ ID NO: 16), PFOA V5 (SEQ ID NO: 17) and PFHxS V5 (SEQ ID NO: 23), each bind to more than one of the PFAS compounds of PFNA, PFOS, PFBS, PFOA, PFHxS and GenX.

TABLE 20
Binding of Antibodies to PFAS Compounds

| VHH Antibody | PFNA | PFOS | PFBS | PFOA | PFHxS | GenX |
|---|---|---|---|---|---|---|
| PFNA V1 | ✓ | ✓ | ✓ | ✓ | ✓ | |
| PFNA V2 | ✓ | | | | | |
| PFNA V3 | ✓ | ✓ | | | | |
| PFNA V4 | ✓ | | | | | |
| PFNA V5 | ✓ | | | | | |
| PFNA V6 | ✓ | | | | | |
| PFNA V7 | ✓ | | | | | |
| PFNA V8 | ✓ | | | | | |
| PFOS V1 | | ✓ | | | | |
| PFOS V2 | | ✓ | | | | |
| PFBS V1 | | | ✓ | | | |
| PFBS V2 | | ✓ | ✓ | ✓ | ✓ | ✓ |
| PFBS V3 | | | ✓ | | | |
| PFOA V1 | | | ✓ | ✓ | | |
| PFOA V2 | | | ✓ | ✓ | | |
| PFOA V3 | | | ✓ | ✓ | | |
| PFOA V5 | | | ✓ | ✓ | | |
| PFOA V6 | | | | ✓ | | |
| PFHxS V1 | | | | | ✓ | |
| PFHxS V2 | | | | | ✓ | |
| PFHxS V3 | | | | | ✓ | |
| PFHxSV4 | | | | | ✓ | |
| PFHxS V5 | | | | | ✓ | ✓ |
| PFHxS V6 | | | | | ✓ | |
| GenX V1 | | | | | | ✓ |
| GenX V2 | | | | | | ✓ |
| GenX V3 | | | | | | ✓ |

One or more of the VHH antibodies which bind to more than one PFAS compound may be useful for inclusion in a broad PFAS compound survey kit configured for use with the detection systems described herein, while the remaining VHH antibodies may be useful for confirming the specific presence of specific PFAS compounds.

### Example 2: Confirmation of Binding of an Anti-PFNA Antibody to PFNA in an ELISA Assay

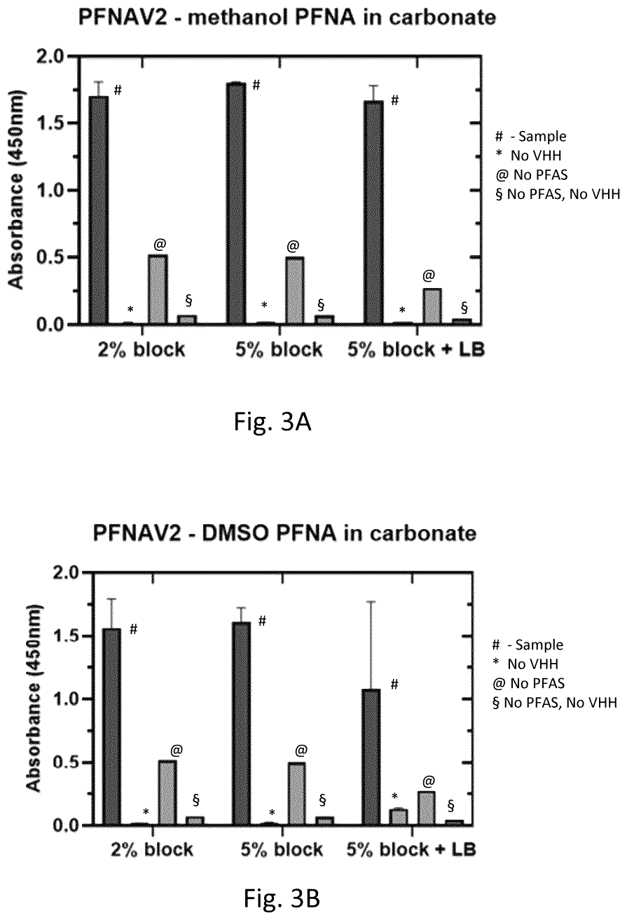

Solutions of PFNA solubilized using methanol or DMSO were diluted in 100 mM of carbonate buffer to prepare solutions of (NaHCO₃12.5 mM, Na2CO3 87.5 mM), pH 10.2 at a final concentration of 20 μg/mL. These solutions were used to coat 96 well flat bottom MaxiSorp™ Nunc-Immuno plates to provide immobilized PFNA. After the coating, and following each subsequent step, plates were washed three times with 0.01 M phosphate buffered saline (PBS, 138 mM NaCl, 2.7 mM KCl, pH 7.4) containing 0.05% Tween 20 (PBST). Unbound sites on the plates were blocked with 5% skim milk prepared in PBS. Affinity purified VHH diluted in PBS or PBS containing LB media (final concentration of 28%) was then added to the wells and incubated for two hours. This was followed by peroxidase conjugated affinity purified anti-hemagglutinin IgG diluted 1:10,000 in 2.5% skim milk in PBS. 100 μL of 3,3′,5,5′-tetramethylbenzidine (TMB) substrate (a conventional peroxidase substrate) was then added for 3 min followed by the equal amount of 2% HCl to stop the reaction and color development generated from the peroxidase activity. Plates were read at 450 nm in a spectrophotometric ELISA microplate reader and values shown are averages of duplicate wells. Data for binding of the VHH antibody PFNA V2 are shown in FIGS. 3A and 3B. The results indicate binding of the antibody PFNA V2 to PFNA at higher levels for the methanol solubilized PFNA coated plates. In contrast, the controls measured in the absence of VHH antibodies, the absence of a PFAS compound and both, did not show significant absorbance at 450 nm.

This example confirms the basis for using an antibody generated against a PFAS compound as a biosensor for detection of a PFAS compound.

### Example 3: Competitive ELISA for Detection of PFOA and PFNA

This example describes a competitive ELISA system with PFAS compound adsorbed directly to the surface of ELISA plates, which competes with free PFAS compound in a sample being analyzed. During the assay, if the His-tagged VHH antibody binds to the PFAS compound adsorbed on the plate, and the secondary anti-His IgG HRP-linked antibody binds to the His-tagged VHH, the resulting antibody complex will remain immobilized during a plate washing step to remove unbound components from the plate. Addition of a peroxidase substrate will the generate an absorbance signal resulting from the peroxidase reaction with the substrate. A drop in absorbance occurs when free PFAS compound in solution competes for binding of the VHH antibody to the immobilized PFAS compound.

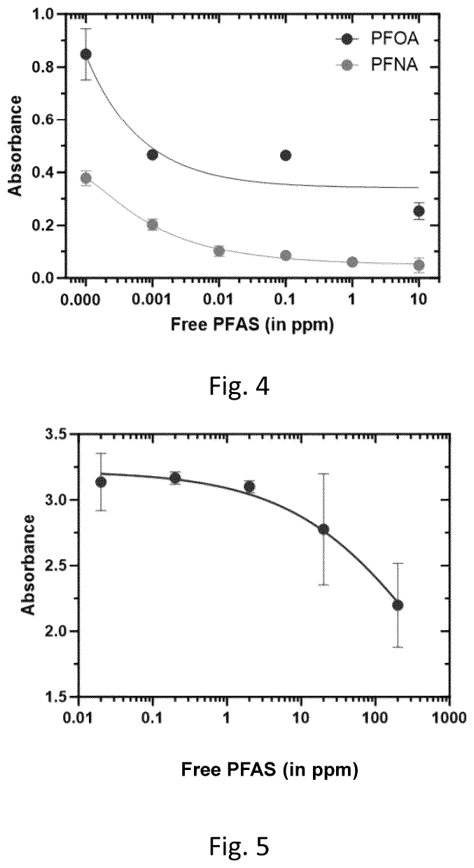

A set of 96 well flat bottom MaxiSorp™ Nunc-Immuno™ plates was coated with methanol-solubilized PFNA, diluted in 100 mM of carbonate buffer (12.5 mM NaHCO₃, 87.5 mM Na2CO3, pH 8.2) at a final concentration of 10 or 50 μg/mL. After the coating, and following each subsequent step, plates were washed three times with PBS (138 mM NaCl, 2.7 mM KCl 2.7 mM, pH 7.4) containing 0.05% Tween 20 (PBST). Unbound sites on the plates were blocked with 2% skim milk prepared in PBS. Affinity purified His-tagged VHH (PFNA V2) diluted in PBS (final concentration of 135 nM) and test samples containing different concentrations of PFOA or PFNA were then added to the wells with incubation for two hours. This was followed by addition of affinity-purified anti-His IgG antibody diluted 1:400 in PBS with one hour of incubation at room temperature, followed by addition of peroxidase conjugated anti-mouse IgG HRP at a final concentration of 1:500. Then 100 μL of ABTS with hydrogen peroxide substrate was added with incubation for 90 min. Plates were read at 415 nm in an EIA plate reader. The results are shown in FIG. 4 and values shown are averages of duplicate wells. The drop in absorbance observed at higher concentrations of PFAS compound provides an indication of the concentration of free PFAS in the sample.

### Example 4: Generation and Selection of VHH Antibodies Recognizing PFOA Using an Alternative ELISA Process

Modified streptavidin-biotin VHH ELISA—A second set of VHH antibodies was generated against PFOA according to the processes outlined in Example 1, with the exception that a modified ELISA assay was used as described below. The modified ELISA was developed because the inventors recognized that the ELISA system described above in Example 1, which has a PFAS molecule adsorbed directly to the surface of the ELISA assay plates might not be sufficiently robust for incorporation into a biosensor system and that assay improvements should be investigated. In a first alternative competitive ELISA assay which uses the streptavidin-biotin interaction, the ELISA assay plates were coated with streptavidin and PFOA was conjugated to biotin via an 11-mer of polyethylene glycol (PEG11) to provide a conjugate designated “biotin-PEG11-PFOA.” Addition of the biotin-PEG11-PFOA conjugate to the streptavidin coated plates provides an immobilized PFOA with increased stability for the competitive ELISA wherein free PFOA (or other PFAS compounds in a sample being analyzed) will compete with the immobilized PFOA. In this ELISA, a hexahistidine tag is conjugated to the VHH antibody to provide a His-tagged VHH antibody. The His-tag provides recognition site for a secondary anti-His IgG antibody, which in turn, is recognized by a tertiary anti-IgG antibody conjugated to horseradish peroxidase (the latter which is provided to increase the signal as a result of binding to additional sites on the anti-His secondary antibody). During the assay, if the His-tagged VHH antibody binds to the immobilized PFOA, and the secondary and tertiary antibodies bind to the His-tagged VHH, the entire antibody complex will remain immobilized during a plate washing step to remove unbound components from the plate. Addition of a peroxidase substrate will the generate an absorbance signal resulting from the peroxidase reaction with the substrate. In testing of an aqueous sample suspected of containing PFOA, the sample is added to the ELISA plate with the bound antibody complex. The free PFOA compound competes with the bound PFOA for binding to the His-tagged VHH. Following washing of the plate to remove unbound components, including His-tagged VHH bound to free PFOA, a drop in absorbance will be measured which correlates with the concentration of PFOA in the sample.

In this competitive ELISA, 96 well flat bottom MaxiSorp™ Nunc-Immuno™ plates were coated with streptavidin, diluted in 100 mM of carbonate buffer (12.5 mM NaHCO₃12.5 mM, Na2CO3 87.5 mM, pH 8.2), at a final concentration of 10 μg/mL. After the coating, and following each subsequent step, plates were washed three times with PBS (138 NaCl mM, 2.7 mM KCl 2.7, pH 7.4). Unbound sites on the plates were blocked with 0.1% casein sodium salt prepared in protein free buffer (PFB, catalog number: 37572) for 1 hour. Biotin-PEG11-PFOA, diluted in PBS pH 7.4 at a final concentration of 200 ppm, was then added to the wells with incubation for 16 hours at room temperature. Five samples of affinity-purified VHH diluted in PBS (final concentration of 200 nM) with different concentrations of biotin-PEG11-PFOA (used as an experimental surrogate for free PFOA), were then added to the wells and the plates were incubated for two hours. This was followed by addition of affinity purified anti-His IgG diluted 1:500 in PBS and one hour of incubation at room temperature. This was followed by addition of peroxidase conjugated anti-mouse IgG HRP step at a final concentration of 1:1,000. Then 100 μL of ABTS with hydrogen peroxide (1:1,000) substrate was then added and the plates were incubated for 30 min. Plates were read at 410 nm in an E1A plate reader and values shown are averages of duplicate wells. The results of a test analysis of 5 samples having different concentrations of biotin-PEG11-PFOA are shown in FIG. 5, where it is seen that absorbance at 410 nm decreases with increasing concentration of biotin-PEG11-PFOA in the test sample.

Results of ELISA—the ELISA results identified a set of four VHH antibodies which bind to PFOA. The amino acid sequences of the VHH antibodies are shown in Table 21, where VHH antibody name designations and sequence identifiers are listed. DNA sequences encoding the VHH antibodies are shown in Table 22. CDR sequences of the VHH antibodies are listed in Table 23.

TABLE 21
Amino Acid Sequences of Second Set of Antibodies

| Anti- | | SEQ |
|---|---|---|
| body | | ID |
| name | Amino acid sequence | NO: |
| BPL1 | DVQLQESGGGSVQAGRSLRLSCATSGYTRGTFRSN | 123 |
| | CMGWFRQGPRKEREGVAAIYTGEGNTYYDDSVKGR | |
| | FTISQDDSKNTMYLQMNSLKPEDTAIYYCAARGGYCS | |
| | DTYWRWDDYNYWGQGTQVTVSS | |
| BPL2 | DVQLQESGGGAVQVGGSLRLSCTASRVTSRDTMAW | 124 |
| | FRQAPGKEREGVATVYPGTAETYYANSVKTRFTISLD | |
| | KTKNMLFLQMNNLKPEDTGMYYCAQAARRAVFGRIA | |
| | LEDENYSDWGQGTQVTVSS | |
| BPL3 | DVQLQESGGGSVQAGGSLRLSCVASGATYCTGDMS | 125 |
| | WYRQAPGNECELVSTISSDGSTYYADSVKGRFTISRD | |
| | NDNATIYLQMNGLKPEDTAKYVCAAQSGPYCYRPLH | |
| | PSEYNKWGQGTQVTVSS | |
| BPL4 | DVQLQESGGGSVEAGGSLTLTCVASEFRSMMAWYR | 126 |
| | QAPGKECEFLSRIPSDGVPVYGDSMKGRSSISRDNA | |
| | KNTVTLQLNSLKVEDTAMYYCASTWAASGGNCPKP | |
| | WDYWGQGTQVTVSSAAAAHHHHHHGAEQKLISEED | |
| | LS | |

TABLE 22
DNA Sequences Encoding Second Set of Antibodies

| Anti- | | SEQ |
|---|---|---|
| body | | ID |
| name | DNA sequence | NO: |
| BPL1 | GATGTGCAGCTGCAGGAGTCTGGGGGAGGCTCGG | 127 |
| | TGCAGGCTGGAAGGTCTCTGAGACTCTCCTGTGCA | |
| | ACCTCTGGGTACACCCGCGGTACCTTCCGTAGCAA | |
| | CTGCATGGGGTGGTTCCGCCAGGGTCCACGGAAG | |
| | GAGCGCGAGGGGGTCGCAGCTATCTATACCGGTG | |
| | AGGGTAACACGTACTATGACGACTCCGTGAAGGGC | |
| | CGATTCACCATCTCCCAAGACGACTCCAAGAACAC | |
| | GATGTATCTGCAGATGAACAGCCTAAAACCTGAGG | |
| | ACACTGCCATTTACTACTGTGCGGCTAGAGGCGGA | |
| | TATTGTAGTGATACTTACTGGCGCTGGGATGACTAT | |
| | AACTACTGGGGCCAGGGGACCCAGGTCACCGTCT | |
| | CCTCAGCGGCCGCGGCACACCATCACCACCATCAT | |
| | GGCGCAGAACAAAAACTCATCTCAGAAGAGGATCT | |
| | GTCTTAG | |
| BPL2 | CAGATGTGCAGCTGCAGGAGTCTGGGGGAGGCGC | 128 |
| | GGTGCAGGTTGGAGGCTCTCTGAGACTCTCCTGTA | |
| | CAGCCTCAAGAGTCACCAGTAGAGACACCATGGCC | |
| | TGGTTCCGCCAGGCTCCAGGGAAGGAGCGCGAGG | |
| | GGGTCGCGACAGTTTATCCTGGCACGGCAGAAACA | |
| | TACTATGCCAACTCCGTGAAGACCCGATTCACCATC | |
| | TCCTTAGACAAGACCAAGAATATGCTATTTCTCCAA | |
| | ATGAATAACCTGAAACCTGAGGACACTGGTATGTAC | |
| | TATTGTGCACAAGCAGCGCGGAGGGCCGTTTTTGG | |
| | ACGCATTGCGTTGGAAGACGAGAACTACTCCGACT | |
| | GGGGCCAGGGGACCCAGGTCACCGTCTCCTCAGC | |
| | GGCCGCGGCACACCATCACCACCATCATGGCGCA | |
| | GAACAAAAACTCATCTCAGAAGAGGATCTGTCTTAG | |
| BPL3 | AGATGTGCAGCTGCAGGAGTCTGGGGGAGGCTCG | 129 |
| | GTGCAGGCTGGAGGATCTCTGAGACTCTCCTGTGT | |
| | AGCCTCTGGAGCCACCTACTGTACTGGAGACATGA | |
| | GCTGGTACCGCCAGGCTCCAGGGAATGAGTGCGA | |
| | GTTGGTCTCAACTATTAGTAGTGATGGTAGCACATA | |
| | CTATGCAGACTCCGTGAAGGGCCGATTCACCATCT | |
| | CCCGAGACAACGACAATGCCACGATATATCTCCAA | |
| | ATGAACGGCCTAAAACCTGAGGACACTGCCAAATA | |
| | CGTCTGTGCTGCTCAGTCGGGGCCGTATTGTTATC | |
| | GTCCCCTTCACCCGTCGGAATATAACAAATGGGGT | |
| | CAGGGGACCCAGGTCACCGTCTCCTCAGCGGCCG | |
| | CGGCACACCATCACCACCATCATGGCGCAGAACAA | |
| | AAACTCATCTCAGAAGAGGATCTGTCTTAG | |
| BPL4 | GATGTGCAGCTGCAGGAGTCTGGGGGAGGCTCGG | 130 |
| | TGGAGGCTGGAGGATCTCTGACACTCACCTGTGTA | |
| | GCCTCTGAATTTAGGTCCATGATGGCATGGTATCGT | |
| | CAGGCCCCAGGGAAGGAGTGCGAATTTCTCTCTCG | |
| | GATTCCTAGTGATGGTGTCCCTGTGTATGGCGACT | |
| | CCATGAAGGGCCGATCGAGCATCTCCCGAGACAAC | |
| | GCCAAGAATACGGTGACCCTTCAGTTGAACAGTCT | |
| | GAAAGTTGAGGATACGGCCATGTATTACTGCGCAT | |
| | CAACCTGGGGGGCCTCCGGAGGTAATTGCCCTAAG | |
| | CCTTGGGATTACTGGGGCCAGGGGACCCAGGTCA | |
| | CCGTCTCCTCAGCGGCCGCGGCACACCATCACCA | |
| | CCATCATGGCGCAGAACAAAAACTCATCTCAGAAG | |
| | AGGATCTGTCTTAG | |

TABLE 23
CDR Sequences of Second Set of Antibodies

| | | | SEQ |
|---|---|---|---|
| Antibody | | | ID |
| name | CDR No: | CDR Sequence | NO: |
| BPL1 | CDR1 | GYTRGTFRSNCMG | 131 |
| BPL1 | CDR2 | AIYTGEGNTY | 132 |
| BPL1 | CDR3 | RGGYCSDTYWRWDDYNY | 133 |
| BPL1 | CDR4 | DSKNTM | 134 |
| BPL2 | CDR1 | RVTSRDTMA | 135 |
| BPL2 | CDR2 | TVYPGTAETY | 136 |
| BPL2 | CDR3 | AARRAVFGRIALEDENYSD | 137 |
| BPL2 | CDR4 | DKTKNM | 138 |
| BPL3 | CDR1 | GATYCTGDMS | 139 |
| BPL3 | CDR2 | TISSDGSTYYADS | 140 |
| BPL3 | CDR3 | QSGPYCYRPLHPSEYNK | 141 |
| BPL3 | CDR4 | DNATIY | 142 |
| BPL4 | CDR1 | EFRSMMA | 143 |
| BPL4 | CDR2 | RIPSDGVPVYGDS | 144 |
| BPL4 | CDR3 | TWAASGGNCPKPWDY | 145 |
| BPL4 | CDR4 | DNAKNT | 59 |

### Example 5: Testing of Binding of the Complete Collection of VHH Antibodies in Direct ELISA

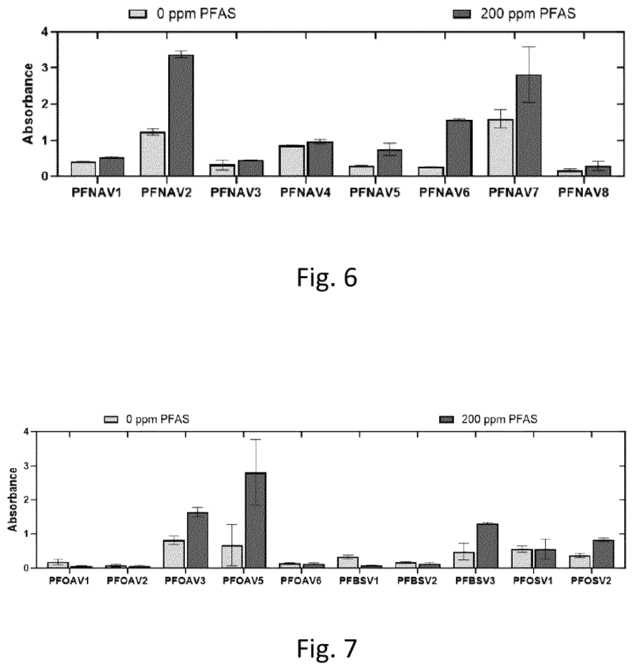

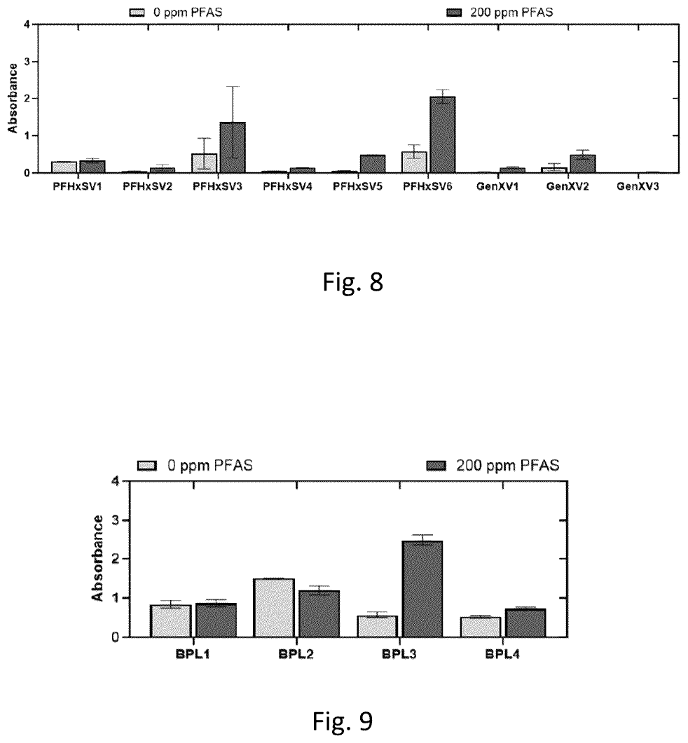

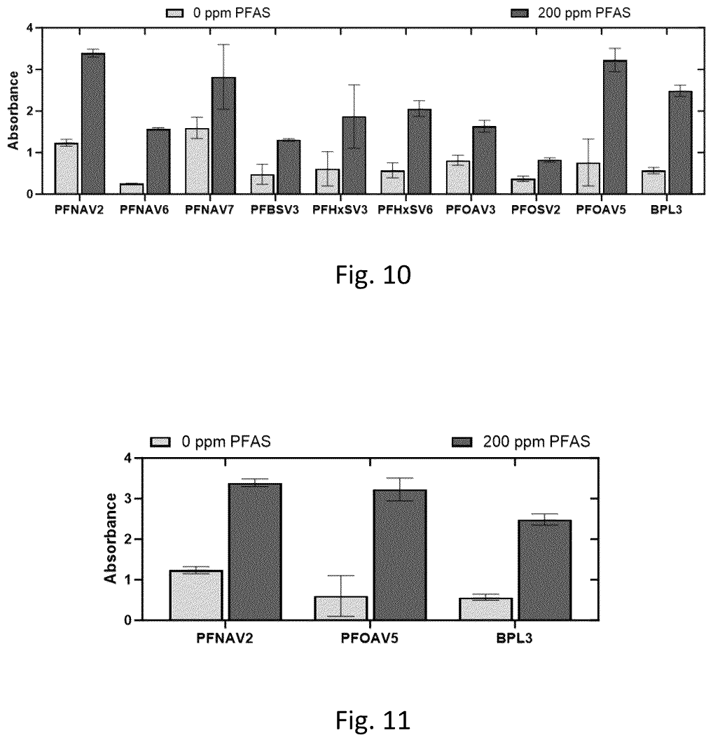

In this experiment, the complete collection of VHH antibodies (including the first set of 27 VHH antibodies and the second set of 4 VHH antibodies were tested in a direct ELISA system for the ability to bind to biotin-PEG11-PFOA. Since there is no competition with free PFAS in this experiment, effective binding of a given VHH antibody is indicated by an increase in absorbance in the assay. A set of 96 well flat bottom MaxiSorp™ Nunc-Immuno™ plates was coated with streptavidin, diluted in 100 mM of carbonate buffer (12.5 mM NaHCO₃, 87.5 mM, pH 8.2) at a final concentration of 10 μg/mL. After the coating, and following each subsequent step, plates were washed three times with PBS (138 mM NaCl, 2.7 KCl mM, pH 7.4). Unbound sites on the plates were blocked with 0.1% casein sodium salt prepared in protein free buffer (PFB, catalog number: 37572) for 1 hour. Biotin-PEG11-PFOA, diluted in PBS pH 7.4 at a final concentration of 0 or 200 ppm, was then added to the wells with incubation for 16 hours at room temperature. Affinity purified VHH diluted in PBS (final concentration of 200 nM), was then added to the wells, followed by incubation for two hours. This was followed by addition of affinity purified anti-His IgG diluted 1:500 in PBS with incubation at room temperature, followed by addition of peroxidase conjugated anti-mouse IgG HRP step at a final concentration of 1:1,000. 100 μL of ABTS with hydrogen peroxide (1:1,000) substrate was then added followed by incubation for 30 min. Plates were read at 410 nm in an E1A plate reader and values shown in the bar charts of FIGS. 6 to 10 are averages of at least duplicate wells. FIG. 10 shows a comparison of the 10 VHH antibodies exhibiting the most effective binding to immobilized PFOA in the direct ELISA and FIG. 11 shows a comparison of the 3 VHH antibodies exhibiting the most effective binding to immobilized PFOA in the direct ELISA.

The results of this experiment indicate that the most effective antibodies in terms of binding to PFOA in this direct ELISA assay are PFNA V2 (SEQ ID NO: 2), PFOA V5 (SEQ ID NO: 17) and BPL3 (SEQ ID NO: 125), as assessed by the increase in absorbance from baseline to 200 ppm PFAS. Other antibodies showing favorable binding properties for binding to PFOA are PFNA V7 (SEQ ID NO: 7), PFHxSV3 (SEQ ID NO: 21) and PFHXSV6 (SEQ ID NO: 24).

### Example 6: Cyclodextrin-Modified Competitive ELISA

It is conventionally known that PFAS compounds have limited solubility and that cyclodextrins can be used to sequester PFAS compounds. The inventors conceived the concept to improve the competitive ELISA described above which uses biotin-PEG11-PFOA by incorporation of a cyclodextrin to bind to free PFAS compounds in solution to improve their solubility to increase the ability of the VHH antibodies to detect PFAS compounds in the ELISA. β-cyclodextrin was selected for this modified assay. It was determined that in the absence of free PFOA, the cyclodextrin binds to the immobilized PFOA and blocks the binding of the His-tagged VHH antibody to the immobilized PFAS. After washing, there is no appreciable amount of immobilized VHH antibody detectable in the peroxidase reaction due to a lack of formation of the tertiary antibody complex and the absorbance level at 410 nm is low. When increasingly higher concentrations of free PFAS are included in test solutions, the cyclodextrin in the solution will bind to the free PFAS molecules in solution and this results in fewer immobilized PFOA sites binding the cyclodextrin and more binding of the His-tagged VHH antibodies, which in turn causes a greater amount of formation of the detectable tertiary antibody complex with the increased absorption measured at 410 nm.

In one experiment, which investigated the three VHH antibodies determined to have the most effective binding to immobilized PFOA in the direct ELISA assay described in Example 5, the cyclodextrin-modified competitive ELISA was performed over a range of PFOA concentrations from under 0.1 ppm to 1000 ppm for BPL3, PFNA V2 and PFOA V5. A set of 96 well flat bottom MaxiSorp™ Nunc-Immuno™ plates were coated with streptavidin, diluted in 100 mM of carbonate buffer (12.5 mM NaHCO₃, 87.5 mM Na2CO3 mM, pH 8.2) at a final concentration of 10 μg/mL. After the coating, and following each subsequent step, plates were washed three times with PBS (138 mM NaCl, 12.7 mM KCl 2.7 mM, pH 7.4). Unbound sites on the plates were blocked with 0.1% casein sodium salt prepared in protein free buffer (PFB, catalog number: 37572) for 1 hour. Biotin-PEG11-PFOA, diluted in PBS pH 7.4 was then added to the wells to a final concentration of 200 ppm, followed by incubation for 16 hours at room temperature. The competition solution including affinity purified VHH diluted in 0.5×PBS (final concentration of 200 nM), 1.4 ppm of β-cyclodextrin was then added with varying concentrations of free PFOA from a methanol diluted stock solution, followed by incubation for two hours. This was followed by addition of affinity purified anti-HIS IgG diluted 1:500 in PBS with incubation for one hour at room temperature followed by addition of peroxidase conjugated anti-mouse IgG HRP step at a final concentration of 1:1,000. 100 μL of ABTS with hydrogen peroxide (1:1,000) substrate was then added for 30 min. Plates were read at 410 nm in an EIA plate reader and values shown are averages of duplicate wells.

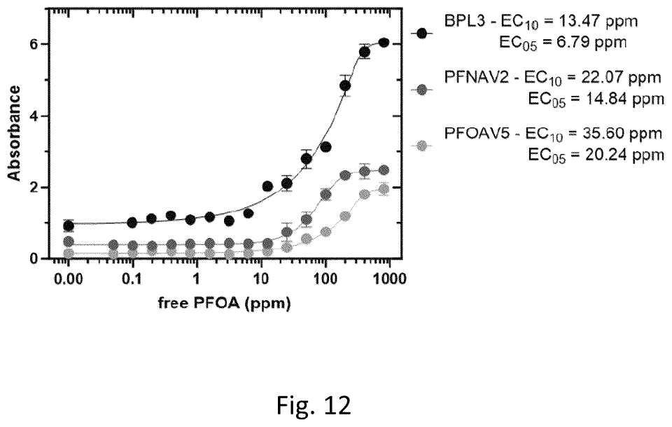

The results of this experiment are shown in FIG. 12. The EC10 and EC05 values shown on FIG. 12 for each of the three antibodies indicate the concentration at which 10% and 5% exhibit a significant improvement from the baseline. VHH antibody BPL3 has the best detection sensitivity with EC10=13.47 ppm and EC05=6.79 ppm. VHH antibody PFNA V2 has the second best detection sensitivity with EC10=22.07 ppm and EC05=14.84 ppm. The remaining VHH antibody PFOA V5 has EC10=35.60 ppm and EC05=20.24 ppm. All three of these VHH antibodies are appropriate for use in a PFAS detection system.

A notable observation was that the VHH antibody BPL3 does not provide the best data in FIG. 11 in comparison with PFNA V2 and PFOA V5 in terms of the difference in absorbance between the 0 ppm and 200 ppm measurements in the direct ELISA. However, BPL3 has the best standard curve in FIG. 12 in the competitive ELISA. Therefore, while BPL3 currently appears to be the most effective VHH antibody for binding of PFAS in view of current data, further testing of the other VHH antibodies may reveal that other VHH antibodies disclosed herein may be even more effective in binding free PFAS molecules in the competitive cyclodextrin ELISA and provide the basis for a biosensor for detection of PFAS molecules in samples. Therefore, it is to be understood that any one of the 31 VHH antibodies disclosed herein will likely be found to be appropriate for incorporation into a PFAS detection system. Furthermore, based on the results of the first set of VHH antibodies described above, it is reasonably predicted that the VHH antibodies of the second set (BPL1, PBL2, BPL3 and PBL4; (SEQ ID NOs: 127 to 130) will be found to have the ability to bind to PFAS compounds other than PFOA with sufficient affinity to use them for detection of the other PFAS compounds.

### EQUIVALENTS AND SCOPE

While various embodiments of the disclosure have been particularly shown and described in the present disclosure, it will be understood by those skilled in the art that various changes in form and details may be made without departing from the scope of the embodiments disclosed herein and set forth in the appended claims.

Those skilled in the art will recognize or be able to ascertain using no more than routine experimentation, many equivalents to the specific embodiments described herein. The scope of the present disclosure is not intended to be limited to the above description, but rather is as set forth in the appended claims.

In the claims, articles such as “a,” “an,” and ‘the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of a group is present in, employed in, or otherwise relevant to a given product or process. The disclosure includes embodiments in which more than one, or all group members are present in, employed in, or otherwise relevant to a given product or process.

It is also noted that the term “comprising” is intended to be open and permits but does not require the inclusion of additional elements or steps. When the term “comprising” is used herein, the terms “consisting of’ and “or including” are thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to those of ordinary skill in the art, they may be excluded even if the exclusion is not set forth explicitly herein. Any particular embodiments of compositions disclosed herein can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the present application, the statement in the present application shall control.

1. An antibody or antibody fragment which binds to perfluorooctanoic acid (PFOA), comprising:
a CDR1 amino acid sequence having at least about 80% sequence identity to SEQ ID NO: 139;
a CDR2 amino acid sequence having at least about 80% sequence identity to SEQ ID NO: 140; and
a CDR3 amino acid sequence having at least about 80% sequence identity to SEQ ID NO: 141.

2. The antibody of claim 1, comprising a CDR4 amino acid sequence having at least about 80% sequence identity to a sequence selected from the group consisting of SEQ ID NO: 142.

3. The antibody or antibody fragment of claim 1, comprising a VHH antibody sequence having at least about 80% sequence identity with SEQ ID NO: 129.

4. The antibody or antibody fragment of claim 1, which binds to one or more additional PFAS compounds selected from the group consisting of PFNA, PFOS, PFBS, PFHxS, and GenX.

5. The antibody or antibody fragment of claim 1, comprising one or more variants of one or more CDRs of the recited CDRs, wherein the one or more variants comprises one or more replacements of hydrophilic amino acid residues with hydrophobic residues, wherein an antibody or antibody fragment comprising the one or more variants exhibits greater binding affinity for a PFAS compound.

6. An isolated nucleic acid sequence encoding the antibody of claim 1.

7. The isolated nucleic acid sequence of claim 6, having the sequence of SEQ ID NO: 129.

8. A cell or virus comprising the isolated nucleic acid sequence of claim 7.

9. A detector system for identification of a PFAS compound comprising the antibody or antibody fragment of claim 1.

10. The detector system of claim 9, wherein the PFAS compound binds to the antibody or antibody fragment in an ELISA assay.

11. The detector system of claim 10, wherein the ELISA assay is a direct ELISA or a competitive ELISA.

12. The detector system of claim 10, wherein the ELISA assay is configured to detect free PFAS compounds in an aqueous sample.

13. The detector system of claim 12, wherein the free PFAS compounds comprise one or more PFAS compounds selected from the group consisting of: PFOA, PFNA, PFOS, PFBS, PFHxS, and GenX.

14. The detector system of claim 11, wherein the ELISA assay comprises an immobilized PFAS compound.

15. The detector system of claim 14, wherein the immobilized PFAS compound is immobilized using a biotin-streptavidin interaction.

16. The detector system of claim 15, wherein the competitive ELISA includes binding of a PFAS molecule conjugated to a biotin moiety.

17. The detector system of claim 16, comprising an assay plate coated with streptavidin, wherein the PFAS molecule conjugated to the biotin moiety provides the immobilized PFAS compound upon binding of the biotin moiety to the streptavidin.

18. The detector system of claim 12, wherein the antibody or antibody fragment includes an affinity tag and a secondary antibody binds to the affinity tag.

19. The detector system of claim 18, comprising a tertiary antibody conjugated to a peroxidase enzyme, wherein the tertiary antibody binds to the secondary antibody, thereby providing a detectable antibody complex via a peroxidase substrate reaction.

20. The detector system of claim 12, wherein the competitive ELISA includes a cyclodextrin which binds to the free PFAS compound and to the immobilized PFAS compound.


SEQUENCE LISTING (145 sequences)

SEQ ID NO: 1 - protein, 123 aa, Camelus dromedarius
DVQLQESGGGSVQMGGSLRLSCAASEFTWETDQMAWFRQAPGQEREPVARIGGGVLQLRTYADYVKGRFTISRDNAKNMVYLQMNSLKPEDTAIYYCAAGPSSTWDIGAYRYWGQGTQVTVSS

SEQ ID NO: 2 - protein, 110 aa, Camelus dromedarius
MGGSLRLSCAASEFTWETDQMAWFRQAPGQEREPVARIGGGVLQLRTYADYVKGRFTISRDNAKNTVYLQMNSLKPEDTAIYYCAAGPSSTWDIGAYRYWGQGTQVTVSS

SEQ ID NO: 3 - protein, 123 aa, Camelus dromedarius
DVQLQESGGGLVQPGGSLRLSCAASEFTWETDQMAWFRQAPGQEREPVARIGGGVLQLRTYADYVKGRFTISRDNAKNMVYLQMNSLKPEDTAIYYCAAGPSSTWDIGAYRYWGQGTQVTVSS

SEQ ID NO: 4 - protein, 123 aa, Camelus dromedarius
DVQLQESGGGLVQPGGSLRLSCAASEFTWETDQMAWFCQAPGQEREPVARIGGGVLQLRTYADYVKGRFTISRDNAKNMVYLQMNSLKPEDTAIYYCAAGPSSTWDIGAYRYWGQGTQVTVSS

SEQ ID NO: 5 - protein, 125 aa, Camelus dromedarius
DVQLQESGGGSVQAGGSLRLSCTVSGYTYSSCSMGWYRQAPGKKRELVSSILTNGTTDYADFVKGRFTISQDNAQNTVYLQMNSLTPEDTAMYYCNNRRAADCSRGGPQYGFMYWGQGTQVTVSS

SEQ ID NO: 6 - protein, 123 aa, Camelus dromedarius
DVQLQESGGGLVQPGGSLRLSCAASEFTWETDQMAWFRQAPGQEREPVARIGGGVLQLRTYADYVKGRFTISRDNAKNMVYLQMNSLKPGDTAIYYCAAGPSSTWDIGAYRYWGQGTQVTVSS

SEQ ID NO: 7 - protein, 118 aa, Camelus dromedarius
DVQLQESGGGLVQPGGSLRLSCAASGFTFNTAKMGWFRQTQGKDCELVSTTWPDGRTNVADSVKGRFTISRDNAKNTLYLQLNSLETEDTAMYYCTLGQWDYGARGYQGQGTQVTVSS

SEQ ID NO: 8 - protein, 124 aa, Camelus dromedarius
DVQLQESGGGSVQAGGSLKLSCAASGFMSSNCMGWFRQAPGKEREGVAVINGVGTTYYANSVKGRFTISRDVAKAKNTIYLQMNSLKSEDTATYYCAADPAGGVCDLSMSFNSWGQGTQVTVSS

SEQ ID NO: 9 - protein, 126 aa, Camelus dromedarius
DVQLQESGGGSVQAGGSLRLSCKASGYIYSSYCMGWFRQAPGKEREGVAAIHSGGGSTVFLDSVRGRFTISRDNARNTVYLQMNSLKPDDTAMYYCATGRAVVPACGERRVGYNYWGQGTQVTVSS

SEQ ID NO: 10 - protein, 123 aa, Camelus dromedarius
DVQLQESGGGSVQAGESLRLTCIIAGCTYCKFEMTWYRQTPTNEREFVAGFDSDGGERYGDSVRGRSSISRDTTKDKFFLEMNNLKPEDAGMYFCKMGSGFCVVTGRDQPEYWGQGTQVTVSS

SEQ ID NO: 11 - protein, 123 aa, Camelus dromedarius
DVQLQESGGGSVQMGGSLRLSCAASEFTWETDQMAWFRQAPGQEREPVARIGGGALQLRTYADYVKGRFTISRDNAKNMVYLQMNSLKPEDTAIYYCAAGPSSTWDIGAYRYWGQGTQVTVSS

SEQ ID NO: 12 - protein, 126 aa, Camelus dromedarius
DVQLQESGGGSVQAGGSLRLSCVFSTYRYCTYDMTWYRQAPGKEREFVSIIDRAGSTTYADSVKGRFTISQDNAKNTVYLQMNSLKPEDTAIYYCAARPPVLGIYCHGEVAMWNYWGQGTQVTVSS

SEQ ID NO: 13 - protein, 123 aa, Camelus dromedarius; identical to SEQ ID NO: 1

SEQ ID NO: 14 - protein, 121 aa, Camelus dromedarius
DVQLQESGGGSVQTGGSLRLSCTAPGFTANMCGMAYFRQTGGNPRVWVASIGTYGDIKYADPVKGRFTISRDSAKDTVYLQMNNLQPEDTGLYTCKANPGVADKCWGYNYWSQGTQVTVSS

SEQ ID NO: 15 - protein, 128 aa, Camelus dromedarius
DVQLQESGGGSVQAGGSLRLSCVASDYTYDFYCMGWFRQVPGKEREGVAVTNKAGVTTFYGDSVKNRFTISQADVENTVYLEMNGLKPEDTAIYYCVLRSSAACPTGSWSSSLNYHTWGRGTQVTVSS

SEQ ID NO: 16 - protein, 128 aa, Camelus dromedarius
DVQLQESGGGSVPAGGSLRLSCAASGYTFSSCCMAWFRQAPGKEREGVASINSGGGTTSYADSVKGRFTISRDNAKNTTYLQMNSLKPEDTAMYYCAAEGPVCGGTYPSLFRGVYNTWGQGTQVTVSS

SEQ ID NO: 17 - protein, 115 aa, Camelus dromedarius
DVQLQESGGGSVQTGGSLRLSCAASGFTFSSHWMYWVRQAPGKGLEWVSTVNWGGGSTYYADSVKGRFTISRDNAKNMLYLQMNSLKPEDTALYYCGEGLDFGYWGQGTQVTVSS

SEQ ID NO: 18 - protein, 123 aa, Camelus dromedarius
DVQLQESGGGLVQPGGSLRLSCAATEFTWETDQMAWFRQAPGQEREPVARIGGGVLQLRTYADYVKGRFTISRDNAKNMVYLQMNSLKPEDTAIYYCAAGPSSTWDIGAYRYWGQGTQVTVSS

SEQ ID NO: 19 - protein, 123 aa, Camelus dromedarius
DVQLQESGGGSVQAGESLRLSCAASGITSGANYMGWFRQAPGKERDWVAVIHFGSGTTYYADSVAGRFTFSQDNAKDTFYLQMDSLKPEVSASYYCATGLVGGGTWSCGYNYWGQGTQVTVSS

SEQ ID NO: 20 - protein, 124 aa, Camelus dromedarius
DVQLQESGGGSVQAGGSLRLSCAASGDRNCKYDMSWYRQAPGKEREFVSVIDNDGRSRYADSVKGRFTISQDNAKSTVYLQMNSLKPEDTAMYYCAAGYPDYSLLLGTPGYDFWGQGTQVTVSS

SEQ ID NO: 21 - protein, 127 aa, Camelus dromedarius
DVQLQESGGGSVQAGGSLRLSCVASGHFHRTYCMGWFRQAPGKEREGVASIFSDGSSTYYADSVKGRFTISQDNAKNTVYLQMNTLQPEDTAIYVCAAETRIDLCNEGSFFTTARYRGQGTQVTVSS

SEQ ID NO: 22 - protein, 115 aa, Camelus dromedarius
DVQLQESGGGLVQPGGSLRLSCAASGFTFTNYYMYWLRQAPGKGLEWVSTINFDGSNTGYANSVKGRFTISRDNAKNTLYLQMNSLKSEDTALYYCATDDVGGERGQGTQVTVSS

SEQ ID NO: 23 - protein, 126 aa, Camelus dromedarius
DVQLQESGGGSVQTGESLRLSCTVSGYTNTKDYIGWFRQAPGKEREGLALFFTDDGTAWFTDSVKDRFALAQENTKNTVSLQMNRLKPSDTAMYFCATNTVGSWSWPLVPTDFHFWGQGTQVTVSS

SEQ ID NO: 24 - protein, 121 aa, Camelus dromedarius
DVQLQESGGGLVQPAGSLRLSCVASGFTFDNYAMGWLRQAPGKEIEWVATINSEGKTSYADSVQDRFIISRDNARNTLYLELNSLKIEDTAKYYCVRGRSPSVLVTTDPPSGQGTQVTVSS

SEQ ID NO: 25 - protein, 123 aa, Camelus dromedarius
DVQLQESGGGLVQPGGSLRLSCAASEFTWETDQMAWFRQAPGQEREPVARIGGGVLQLRTYADYVKGRFTISRDNAKNMVYLQMNSLKPEDTAIYYCAAGPSSTWDIGAYRHWGQGTQVTVSS

SEQ ID NO: 26 - protein, 153 aa, Camelus dromedarius
DVQLQESGGGSVQMGGSLRLSCAASEFTWETDQMAWFRQAPGQEREPVARIGGGVLQLRTYADYVKGRFTISRDNAKNMVYLQMNSLKPEDTAIYYCAAGPSSTWDIGAYRYWGQGTQVTVSSAAADGSSPVSWRATSGYKYWGQGTQVTVSS

SEQ ID NO: 27 - protein, 123 aa, Camelus dromedarius
DVQLQESGGGLVQPGGSLRLSCAASEFTWETDQMAWSRQAPGQEREPVARIGGGVLQLRTYADYVKGRFTISRDNAKNMVYLQMNSLKPEDTAIYYCAAGPSSTWDIGAYRYWGQGTQVTVSS

SEQ ID NO: 28 - DNA, 369 bp, synthetic construct
  source 1..369: mol_type = other DNA
gacgtccagctgcaggaatctggcggtggctccgtgcagatgggtggctccctgcgtctgtcttgtgcagcgtccgaattcacctgggaaactgatcagatggcgtggttccgtcaggcgccgggccaggaacgtgaaccggtagctcgtattggcggtggcgttctgcagctgcgcacttatgcagactatgttaaaggccgttttacgattagccgcgataacgccaagaacatggtttacctgcagatgaacagcctgaagccggaggatactgctatttattactgcgcagccggtccgtcttccacttgggacatcggcgcttatcgttactggggccagggcactcaggtgacggtgagctcc

SEQ ID NO: 29 - DNA, 330 bp, synthetic construct
  source 1..330: mol_type = other DNA
atgggtggcagcctgcgtctgagctgcgcggcatctgagtttacgtgggaaactgaccagatggcctggttccgtcaggcgccgggtcaggaacgcgaaccggtggcgcgtatcggtggcggtgttctgcagctgcgtacgtacgcggactacgtaaaaggccgtttcaccatctcccgtgacaacgctaaaaataccgtatatctgcagatgaactctctgaaaccggaagataccgctatctactattgtgctgccggtccgagctccacttgggacattggcgcctatcgttattggggtcaaggtacccaggtaaccgtttccagc

SEQ ID NO: 30 - DNA, 369 bp, synthetic construct
  source 1..369: mol_type = other DNA
gacgtccagctgcaggaatccggtggcggtctggttcaaccgggtggctctctgcgcctgtcctgtgcggctagcgaattcacctgggaaactgatcagatggcatggtttcgccaggctccgggtcaggaacgtgaacctgtagcacgcatcggtggcggtgtcctgcagctgcgtacctacgccgattacgttaaaggccgcttcaccatctctcgtgacaacgcgaaaaacatggtatacctgcaaatgaacagcctgaagccggaagatacggccatctattactgtgcggctggcccgagctccacttgggatattggcgcgtaccgttactggggtcagggtacccaagtaacggtaagctct

SEQ ID NO: 31 - DNA, 369 bp, synthetic construct
  source 1..369: mol_type = other DNA
gacgtccaactgcaggaaagcggtggcggtctggttcagccaggtggctccctgcgtctcagctgcgcggcctctgaattcacttgggaaaccgatcagatggcctggttctgccaggcgccaggtcaggagcgtgagccggttgctcgcatcggcggtggcgtactgcagctgcgtacttacgcggattatgtgaaaggtcgctttactatctctcgcgataacgctaaaaatatggtgtacctgcagatgaactccctgaagccggaagacacggctatctattactgcgcagccggtccgtccagcacttgggacatcggtgcttatcgttactggggccagggcactcaggtgactgtcagcagc

SEQ ID NO: 32 - DNA, 375 bp, synthetic construct
  source 1..375: mol_type = other DNA
gacgtacagctgcaggaaagcggcggtggcagcgttcaagctggcggttctctgcgcctgtcttgcaccgtgtccggctacacctactcctcttgctctatgggctggtaccgccaggcaccgggtaaaaagcgtgaactggtttctagcattctgaccaacggtactaccgattatgccgacttcgtaaaaggccgttttaccatctctcaggacaacgcgcagaataccgtgtacctgcagatgaacagcctgaccccggaagatacggcaatgtattactgcaataaccgtcgcgcagctgactgttcccgtggcggtccacagtacggctttatgtattggggccagggcacgcaggtaacggtttctagc

SEQ ID NO: 33 - DNA, 369 bp, synthetic construct
  source 1..369: mol_type = other DNA
gatgtccagctgcaggaaagcggcggtggcctggttcagccgggcggttccctgcgcctgagctgcgctgcgtctgaattcacctgggaaaccgatcaaatggcgtggttccgccaggccccaggtcaagagcgtgaaccggtggcgcgtatcggcggtggcgtactgcaactgcgtacctacgcggattacgttaaaggccgtttcaccattagccgtgataacgctaaaaacatggtttacctgcagatgaactccctgaagcctggcgacaccgctatttattactgcgcggccggtccgtccagcacttgggatattggcgcttaccgctactggggtcagggtacgcaggttaccgtatcctcc

SEQ ID NO: 34 - DNA, 354 bp, synthetic construct
  source 1..354: mol_type = other DNA
gacgttcaactccaagaatctggtggcggtctggtgcagccgggcggttctctgcgtctgtcctgtgcggcatccggctttaccttcaacactgctaagatgggttggttccgccagacgcagggcaaagattgtgaactggtcagcacgacctggccggatggtcgtactaatgtggctgattccgtaaaaggccgctttactatctctcgcgacaacgcgaagaacaccctgtatctgcagctgaactccctggaaactgaggacaccgcaatgtattactgtaccctgggtcagtgggattatggtgcgcgtggctatcagggtcagggcacccaagttaccgtgtcctct

SEQ ID NO: 35 - DNA, 372 bp, synthetic construct
  source 1..372: mol_type = other DNA
gatgtacagctgcaggagtctggcggtggcagcgtacaggcgggcggttctctgaaactgtcttgcgcagccagcggcttcatgtctagcaactgcatgggttggtttcgtcaggcaccgggtaaagaacgtgaaggcgtagctgttatcaacggtgttggcactacgtactatgcgaactctgtaaaaggtcgttttacgatttcccgtgatgttgcgaaagcgaagaacaccatctatctgcaaatgaactctctgaaaagcgaagataccgctacctactattgcgctgccgatccggcgggcggtgtctgcgacctgtctatgtctttcaacagctggggtcagggtacccaggttacggttagcagc

SEQ ID NO: 36 - DNA, 378 bp, synthetic construct
  source 1..378: mol_type = other DNA
gacgtacagctgcaggaatctggtggcggttctgtccaagcgggtggctctctgcgcctgagctgcaaagcgagcggttacatttactcctcttattgtatgggctggttccgtcaagcgccgggtaaagaacgtgaaggtgttgccgcgattcatagcggcggtggctccactgtattcctcgattctgtacgtggtcgtttcactattagccgtgacaacgcccgtaacaccgtctatctgcagatgaactctctgaaaccggatgacactgcgatgtattactgcgcgaccggtcgcgctgtagttcctgcttgtggtgagcgtcgcgttggctataactattggggccagggtactcaggttaccgtttctagc

SEQ ID NO: 37 - DNA, 369 bp, synthetic construct
  source 1..369: mol_type = other DNA
gacgtccagctgcaggaatctggtggcggttccgttcaggcaggtgagtccctgcgtctgacttgcattatcgctggctgcacttactgcaaatttgaaatgacctggtaccgtcagactccgaccaacgaacgcgaatttgttgcgggtttcgatagcgacggtggcgaacgttacggtgattctgttcgtggtcgctcctctattagccgtgatactaccaaggacaagttctttctggaaatgaataacctgaaaccggaagatgctggcatgtacttctgcaaaatgggctccggcttctgcgtggtcactggccgtgaccagccggaatattggggtcagggcacgcaggtaacggtttcctct

SEQ ID NO: 38 - DNA, 369 bp, synthetic construct
  source 1..369: mol_type = other DNA
gatgtccagctgcaagagtccggtggcggttccgttcaaatgggcggttccctgcgcctgtcttgtgctgcatccgagttcacttgggaaaccgatcagatggcgtggttccgtcaggctccgggtcaggaacgtgagccggttgctcgtattggcggtggcgctctgcagctgcgtacctatgcagactatgtgaaaggtcgctttaccattagccgcgacaacgctaaaaacatggtgtacctgcagatgaactccctgaaaccggaagataccgccatttattactgcgcggctggtccatcttccacgtgggacattggcgcctaccgttactggggtcagggcacccaggtcactgtttcttct

SEQ ID NO: 39 - DNA, 378 bp, synthetic construct
  source 1..378: mol_type = other DNA
gacgtccagctccaggaatctggtggcggttctgttcaggcgggtggctccctgcgcctgtcttgcgttttcagcacctatcgttactgtacttacgacatgacctggtatcgccaggcgccgggtaaggaacgtgagttcgttagcattatcgaccgcgctggctccactacctacgctgactccgtaaaaggtcgtttcaccatctctcaggacaacgccaaaaacactgtctatctgcagatgaactctctgaagccggaggatacggctatttattactgtgctgcgcgtccgcctgttctgggtatttactgccatggtgaagtggcaatgtggaactactggggtcagggcacccaggtgaccgtttcctct

SEQ ID NO: 40 - DNA, 369 bp, synthetic construct
  source 1..369: mol_type = other DNA
gatgtacagctgcaagaaagcggtggcggttccgtccagatgggtggctctctgcgcctctcttgcgctgcatccgaattcacctgggagactgatcaaatggcatggttccgtcaagcgccgggtcaagaacgtgagccggttgcacgcatcggtggcggtgtgctgcagctgcgtacctacgcagactacgtgaaaggtcgcttcaccatttctcgtgacaacgctaaaaacatggtttacctgcaaatgaactctctcaaacctgaggataccgcgatctattactgtgcggctggtccgagctccacgtgggacattggtgcttatcgttactggggtcagggtacgcaggtgaccgtgagcagc

SEQ ID NO: 41 - DNA, 363 bp, synthetic construct
  source 1..363: mol_type = other DNA
gacgttcaactgcaagaatctggtggcggtagcgtccaaaccggcggttctctccgcctgtcttgcactgcgcctggtttcaccgccaacatgtgcggcatggcctatttccgtcagactggtggcaacccacgcgtttgggtagcctccattggtacctacggcgacattaaatacgcggacccggtgaagggccgtttcaccatctctcgcgattctgcgaaagacactgtgtacctgcagatgaataacctgcaaccggaagacaccggcctgtacacctgcaaagctaacccgggcgtagcagacaaatgctggggttacaactattggtcccagggtacccaagttactgtgtcctct

SEQ ID NO: 42 - DNA, 384 bp, synthetic construct
  source 1..384: mol_type = other DNA
gatgttcagctgcaggagtccggcggtggcagcgtacaagcaggcggttccctgcgcctgtcctgtgtcgcttctgactacacctacgatttttactgcatgggttggttccgccaggttccgggcaaagaacgtgaaggtgtggccgttaccaacaaagcaggtgttacgaccttctacggtgactctgttaaaaaccgcttcaccatcagccaagctgacgtcgagaatactgtttacctggaaatgaacggcctgaaaccggaagacactgcgatctactattgcgtgctgcgttccagcgcagcgtgcccgaccggctcctggtccagctccctcaattaccatacctggggtcgcggcacccaagttaccgtcagctcc

SEQ ID NO: 43 - DNA, 384 bp, synthetic construct
  source 1..384: mol_type = other DNA
gacgtgcaactgcaggaatctggtggcggttctgttccggctggcggttctctgcgtctgagctgcgctgcctccggctataccttttctagctgttgcatggcttggtttcgccaggcgccgggcaaagaacgtgaaggcgtcgcgagcatcaacagcggcggtggcactacctcctacgctgattctgttaagggccgtttcaccatctctcgtgacaacgctaaaaacacgacctacctgcagatgaatagcctgaaaccggaagacactgcaatgtactattgcgcagcggaaggtccggtatgcggcggtacgtatccgagcctgtttcgtggtgtatataacacctggggccagggcacccaggtgacggtttcttct

SEQ ID NO: 44 - DNA, 345 bp, synthetic construct
  source 1..345: mol_type = other DNA
gacgttcagctgcaggaatctggtggcggttctgttcagaccggtggctctctgcgcctgtcttgcgctgcgagcggcttcacctttagctctcactggatgtactgggttcgtcaggccccgggtaaaggcctggaatgggtgtccacggttaactggggtggcggttccacttactatgcagattctgtgaaaggccgcttcaccatttcccgtgataatgcaaaaaacatgctgtacctgcagatgaatagcctgaaaccggaagataccgctctgtattactgcggtgaaggcctggacttcggttactggggccagggcactcaggtgaccgtgtcttct

SEQ ID NO: 45 - DNA, 369 bp, synthetic construct
  source 1..369: mol_type = other DNA
gacgtacaactgcaggaatctggcggtggcctggtccagcctggcggttccctgcgtctcagctgcgctgcgaccgagttcacgtgggaaaccgatcagatggcttggttccgtcaggctccgggtcaggaacgtgaaccggtggcgcgcatcggcggtggcgtactgcagctccgtacctacgcggactacgttaagggtcgcttcaccatctctcgtgacaacgcgaaaaacatggtgtatctgcagatgaattctctgaaaccggaagacaccgctatctattactgcgctgcaggcccgtcctctacctgggacatcggcgcgtaccgttattggggccagggcacccaggttaccgtatccagc

SEQ ID NO: 46 - DNA, 369 bp, synthetic construct
  source 1..369: mol_type = other DNA
gacgtacagctgcaagagagcggcggtggcagcgtgcaggcgggtgagtccctgcgtctgtcttgcgcggcctctggtattacctctggcgctaactacatgggttggtttcgccaggctccgggtaaagaacgtgactgggtagcggttatccattttggctctggtactacctattacgctgacagcgtagctggtcgtttcactttttctcaagacaacgctaaagacactttctacctgcagatggactccctgaaaccggaagtttctgcgtcctactattgcgcgaccggcctggtgggtggcggtacttggagctgcggttataactactggggtcagggcactcaggtaaccgtctcttct

SEQ ID NO: 47 - DNA, 372 bp, synthetic construct
  source 1..372: mol_type = other DNA
gacgtccagctgcaggaatccggcggtggctccgtacaggcgggtggcagcctgcgcctgtcttgcgctgcgtctggtgaccgtaactgtaaatacgatatgtcttggtaccgtcaagcgccaggcaaggaacgcgaattcgtctccgttatcgacaacgatggccgttcccgttatgcagactccgttaaaggccgtttcaccatctctcaagataacgcgaaatctaccgtatacctgcaaatgaattctctcaaaccggaagatactgccatgtactattgcgcggctggttatccggattattctctgctcctgggtacgccaggctatgatttctggggccagggtacccaagttacggtttcctct

SEQ ID NO: 48 - DNA, 381 bp, synthetic construct
  source 1..381: mol_type = other DNA
gatgtgcagctgcaggagtctggcggtggctctgttcaggcgggtggctctctgcgtctgtcttgtgttgcgagcggtcacttccaccgtacctactgcatgggttggttccgtcaagcgccgggcaaggaacgtgaaggtgtggcgagcatcttctctgacggctctagcacgtactatgcggacagcgttaaaggccgtttcaccatcagccaagataacgcaaaaaacaccgtttatctgcagatgaacaccctgcagccggaagacactgctatctacgtttgcgctgcagaaacccgtatcgacctgtgcaatgaaggctctttctttactaccgcacgttatcgtggccagggcacgcaagttaccgtttcctcc

SEQ ID NO: 49 - DNA, 345 bp, synthetic construct
  source 1..345: mol_type = other DNA
gacgttcagctgcaggaaagcggcggtggcctggtccagccaggtggctctctgcgtctgtcctgcgcagcctccggttttaccttcaccaactactatatgtactggctgcgtcaggctccgggtaaaggcctggaatgggtctctactatcaacttcgatggcagcaacactggttacgcgaactctgtaaagggtcgcttcacgatcagccgtgacaatgcgaaaaacaccctgtacctgcagatgaactctctcaagtccgaagacaccgcgctgtattactgtgctaccgacgatgtaggcggtgaacgtggtcagggcacccaagtgaccgtttcttcc

SEQ ID NO: 50 - DNA, 378 bp, synthetic construct
  source 1..378: mol_type = other DNA
gatgttcaactgcaggaaagcggcggtggctccgtacagaccggcgaaagcctgcgcctgagctgcaccgtatccggctacaccaacaccaaagattacatcggctggttccgtcaggctcctggtaaagaacgtgaaggcctggctctgttctttactgacgatggtaccgcctggttcaccgacagcgttaaagatcgcttcgcgctggctcaggaaaacactaaaaacaccgtgagcctgcagatgaaccgtctgaagccttccgacacggccatgtatttttgtgctaccaacaccgttggttcttggtcctggcctctggttccgaccgatttccatttttggggccagggtactcaggttaccgtttctagc

SEQ ID NO: 51 - DNA, 363 bp, synthetic construct
  source 1..363: mol_type = other DNA
gacgtacagctgcaggagtccggtggcggtctggttcagccggcgggttctctgcgcctgtcttgcgttgcatccggcttcacctttgataactacgctatgggttggctgcgccaggcgccgggtaaagaaattgaatgggttgcgaccatcaattccgaaggcaaaaccagctatgcagactccgttcaggaccgcttcattatctcccgtgacaacgcgcgtaatactctgtacctggaactgaacagcctgaaaattgaagataccgcgaaatactattgcgttcgtggccgctctccgtctgtcctggtcacgactgatcctccgagcggccaaggtacgcaagtcaccgtctctagc

SEQ ID NO: 52 - DNA, 369 bp, synthetic construct
  source 1..369: mol_type = other DNA
gacgttcagctgcaggagtctggtggcggtctggtccagccgggtggcagcctgcgcctgagctgtgcggcatctgagtttacctgggaaaccgaccaaatggcatggtttcgtcaggcgccgggtcaggaacgtgagccggtcgcacgtattggcggtggcgtactgcagctgcgtacttatgcagactacgtcaaaggccgtttcaccatcagccgtgacaacgcgaagaacatggtttatctgcagatgaactctctcaaaccggaagataccgcgatctactattgcgcggctggcccgagctctacttgggatatcggtgcgtaccgccactggggccagggtacgcaggtcaccgtgagctct

SEQ ID NO: 53 - DNA, 459 bp, synthetic construct
  source 1..459: mol_type = other DNA
gacgtacagctgcaggagtccggtggcggttccgtgcagatgggcggttccctgcgtctgagctgtgcagctagcgaattcacctgggagactgatcagatggcatggttccgtcaggcgccgggccaagaacgtgagccagtcgctcgtatcggtggcggtgttctgcagctgcgtacctacgcagattatgttaaaggtcgttttacgatttctcgtgacaacgctaaaaacatggtttatctccagatgaacagcctgaagccagaagataccgctatctattactgcgccgcgggcccttccagcacctgggacatcggtgcgtaccgttactggggccagggtactcaggtcactgtttccagcgcggccgctgatggttccagccctgtttcctggcgcgcaacctctggctacaaatactggggccagggtacccaggttactgtgtcctcc

SEQ ID NO: 54 - DNA, 369 bp, synthetic construct
  source 1..369: mol_type = other DNA
gatgtccagctgcaggaatccggtggcggtctggttcagccgggcggtagcctgcgtctgagctgcgcggcttccgagttcacctgggaaaccgaccagatggcgtggagccgtcaggcgccgggccaggaacgcgaaccggtcgcgcgtatcggtggcggtgtgctgcagctgcgcacttacgcagattacgtgaaaggtcgtttcaccattagccgcgataacgcaaaaaacatggtgtatctgcagatgaactctctgaaaccggaagataccgctatctattactgtgcagcgggcccatcctctacctgggacatcggtgcgtaccgttactggggccagggcacccaggtcaccgtctcttcc

SEQ ID NO: 55 - protein, 10 aa, synthetic construct
EFTWETDQMA

SEQ ID NO: 56 - protein, 11 aa, synthetic construct
RIGGGVLQLRT

SEQ ID NO: 57 - protein, 13 aa, synthetic construct
GPSSTWDIGAYRY

SEQ ID NO: 58 - protein, 6 aa, synthetic construct
DNAKNM

SEQ ID NO: 59 - protein, 6 aa, synthetic construct
DNAKNT

SEQ ID NO: 60 - protein, 10 aa, synthetic construct
GYTYSSCSMG

SEQ ID NO: 61 - protein, 9 aa, synthetic construct
SILTNGTTD

SEQ ID NO: 62 - protein, 9 aa, synthetic construct
GGPQYGFMY

SEQ ID NO: 63 - protein, 6 aa, synthetic construct
DNAQNT

SEQ ID NO: 64 - protein, 10 aa, synthetic construct
GFTFNTAKMG

SEQ ID NO: 65 - protein, 9 aa, synthetic construct
TTWPDGRTN

SEQ ID NO: 66 - protein, 10 aa, synthetic construct
GQWDYGARGY

SEQ ID NO: 67 - protein, 9 aa, synthetic construct
GFMSSNCMG

SEQ ID NO: 68 - protein, 9 aa, synthetic construct
VINGVGTTY

SEQ ID NO: 69 - protein, 15 aa, synthetic construct
DPAGGVCDLSMSFNS

SEQ ID NO: 70 - protein, 6 aa, synthetic construct
DVAKAK

SEQ ID NO: 71 - protein, 10 aa, synthetic construct
GYIYSSYCMG

SEQ ID NO: 72 - protein, 10 aa, synthetic construct
AIHSGGGSTV

SEQ ID NO: 73 - protein, 7 aa, synthetic construct
RRVGYNY

SEQ ID NO: 74 - protein, 6 aa, synthetic construct
DNARNT

SEQ ID NO: 75 - protein, 10 aa, synthetic construct
GCTYCKFEMT

SEQ ID NO: 76 - protein, 9 aa, synthetic construct
GFDSDGGER

SEQ ID NO: 77 - protein, 8 aa, synthetic construct
TGRDQPEY

SEQ ID NO: 78 - protein, 6 aa, synthetic construct
DAGMYF

SEQ ID NO: 79 - protein, 10 aa, synthetic construct
TYRYCTYDMT

SEQ ID NO: 80 - protein, 9 aa, synthetic construct
IIDRAGSTT

SEQ ID NO: 81 - protein, 18 aa, synthetic construct
RPPVLGIYCHGEVAMWNY

SEQ ID NO: 82 - protein, 10 aa, synthetic construct
DYTYDFYCMG

SEQ ID NO: 83 - protein, 10 aa, synthetic construct
VTNKAGVTTF

SEQ ID NO: 84 - protein, 11 aa, synthetic construct
GSWSSSLNYHT

SEQ ID NO: 85 - protein, 6 aa, synthetic construct
DTAIYY

SEQ ID NO: 86 - protein, 21 aa, synthetic construct
GFTANMCGMAYFRQTGGNPRV

SEQ ID NO: 87 - protein, 8 aa, synthetic construct
SRDSAKDT

SEQ ID NO: 88 - protein, 13 aa, synthetic construct
NPGVADKCWGYNY

SEQ ID NO: 89 - protein, 6 aa, synthetic construct
DSAKDT

SEQ ID NO: 90 - protein, 10 aa, synthetic construct
GYTFSSCCMA

SEQ ID NO: 91 - protein, 10 aa, synthetic construct
SINSGGGTTS

SEQ ID NO: 92 - protein, 12 aa, synthetic construct
TYPSLFRGVYNT

SEQ ID NO: 93 - protein, 10 aa, synthetic construct
GFTFSSHWMY

SEQ ID NO: 94 - protein, 10 aa, synthetic construct
TVNWGGGSTY

SEQ ID NO: 95 - protein, 6 aa, synthetic construct
GLDFGY

SEQ ID NO: 96 - protein, 10 aa, synthetic construct
GITSGANYMG

SEQ ID NO: 97 - protein, 10 aa, synthetic construct
VIHFGSGTTY

SEQ ID NO: 98 - protein, 14 aa, synthetic construct
GLVGGGTWSCGYNY

SEQ ID NO: 99 - protein, 6 aa, synthetic construct
DNAKDT

SEQ ID NO: 100 - protein, 10 aa, synthetic construct
GDRNCKYDMS

SEQ ID NO: 101 - protein, 9 aa, synthetic construct
VIDNDGRSR

SEQ ID NO: 102 - protein, 16 aa, synthetic construct
GYPDYSLLLGTPGYDF

SEQ ID NO: 103 - protein, 6 aa, synthetic construct
DNAKST

SEQ ID NO: 104 - protein, 10 aa, synthetic construct
GHFHRTYCMG

SEQ ID NO: 105 - protein, 10 aa, synthetic construct
SIFSDGSSTY

SEQ ID NO: 106 - protein, 18 aa, synthetic construct
ETRIDLCNEGSFFTTARY

SEQ ID NO: 107 - protein, 10 aa, synthetic construct
GFTFTNYYMY

SEQ ID NO: 108 - protein, 10 aa, synthetic construct
TINFDGSNTG

SEQ ID NO: 109 - protein, 6 aa, synthetic construct
DDVGGE

SEQ ID NO: 110 - protein, 10 aa, synthetic construct
GYTNTKDYIG

SEQ ID NO: 111 - protein, 10 aa, synthetic construct
LFFTDDGTAW

SEQ ID NO: 112 - protein, 17 aa, synthetic construct
NTVGSWSWPLVPTDFHF

SEQ ID NO: 113 - protein, 6 aa, synthetic construct
DRFALA

SEQ ID NO: 114 - protein, 10 aa, synthetic construct
GFTFDNYAMG

SEQ ID NO: 115 - protein, 9 aa, synthetic construct
TINSEGKTS

SEQ ID NO: 116 - protein, 13 aa, synthetic construct
GRSPSVLVTTDPP

SEQ ID NO: 117 - protein, 13 aa, synthetic construct
GPSSTWDIGAYRH

SEQ ID NO: 118 - DNA, 23 bp, synthetic construct
  source 1..23: mol_type = other DNA
gtcctggctgctcttctacaagg

SEQ ID NO: 119 - DNA, 23 bp, synthetic construct
  source 1..23: mol_type = other DNA
ggtacgtgctgttgaactgttcc

SEQ ID NO: 120 - DNA, 29 bp, synthetic construct
  source 1..29: mol_type = other DNA
gatgtgcagctgcaggagtctggaggagg

SEQ ID NO: 121 - DNA, 29 bp, synthetic construct
  source 1..29: mol_type = other DNA
gatgtgcagctgcaggagtctgggggagg

SEQ ID NO: 122 - DNA, 34 bp, synthetic construct
  source 1..34: mol_type = other DNA
ctagtgcggccgctgaggagacggtgacctgggt

SEQ ID NO: 123 - protein, 129 aa, Camelus dromedarius
DVQLQESGGGSVQAGRSLRLSCATSGYTRGTFRSNCMGWFRQGPRKEREGVAAIYTGEGNTYYDDSVKGRFTISQDDSKNTMYLQMNSLKPEDTAIYYCAARGGYCSDTYWRWDDYNYWGQGTQVTVSS

SEQ ID NO: 124 - protein, 127 aa, Camelus dromedarius
DVQLQESGGGAVQVGGSLRLSCTASRVTSRDTMAWFRQAPGKEREGVATVYPGTAETYYANSVKTRFTISLDKTKNMLFLQMNNLKPEDTGMYYCAQAARRAVFGRIALEDENYSDWGQGTQVTVSS

SEQ ID NO: 125 - protein, 125 aa, Camelus dromedarius
DVQLQESGGGSVQAGGSLRLSCVASGATYCTGDMSWYRQAPGNECELVSTISSDGSTYYADSVKGRFTISRDNDNATIYLQMNGLKPEDTAKYVCAAQSGPYCYRPLHPSEYNKWGQGTQVTVSS

SEQ ID NO: 126 - protein, 143 aa, Camelus dromedarius
DVQLQESGGGSVEAGGSLTLTCVASEFRSMMAWYRQAPGKECEFLSRIPSDGVPVYGDSMKGRSSISRDNAKNTVTLQLNSLKVEDTAMYYCASTWAASGGNCPKPWDYWGQGTQVTVSSAAAAHHHHHHGAEQKLISEEDLS

SEQ ID NO: 127 - DNA, 459 bp, synthetic construct
  source 1..459: mol_type = other DNA
gatgtgcagctgcaggagtctgggggaggctcggtgcaggctggaaggtctctgagactctcctgtgcaacctctgggtacacccgcggtaccttccgtagcaactgcatggggtggttccgccagggtccacggaaggagcgcgagggggtcgcagctatctataccggtgagggtaacacgtactatgacgactccgtgaagggccgattcaccatctcccaagacgactccaagaacacgatgtatctgcagatgaacagcctaaaacctgaggacactgccatttactactgtgcggctagaggcggatattgtagtgatacttactggcgctgggatgactataactactggggccaggggacccaggtcaccgtctcctcagcggccgcggcacaccatcaccaccatcatggcgcagaacaaaaactcatctcagaagaggatctgtcttag

SEQ ID NO: 128 - DNA, 455 bp, synthetic construct
  source 1..455: mol_type = other DNA
cagatgtgcagctgcaggagtctgggggaggcgcggtgcaggttggaggctctctgagactctcctgtacagcctcaagagtcaccagtagagacaccatggcctggttccgccaggctccagggaaggagcgcgagggggtcgcgacagtttatcctggcacggcagaaacatactatgccaactccgtgaagacccgattcaccatctccttagacaagaccaagaatatgctatttctccaaatgaataacctgaaacctgaggacactggtatgtactattgtgcacaagcagcgcggagggccgtttttggacgcattgcgttggaagacgagaactactccgactggggccaggggacccaggtcaccgtctcctcagcggccgcggcacaccatcaccaccatcatggcgcagaacaaaaactcatctcagaagaggatctgtcttag

SEQ ID NO: 129 - DNA, 448 bp, synthetic construct
  source 1..448: mol_type = other DNA
agatgtgcagctgcaggagtctgggggaggctcggtgcaggctggaggatctctgagactctcctgtgtagcctctggagccacctactgtactggagacatgagctggtaccgccaggctccagggaatgagtgcgagttggtctcaactattagtagtgatggtagcacatactatgcagactccgtgaagggccgattcaccatctcccgagacaacgacaatgccacgatatatctccaaatgaacggcctaaaacctgaggacactgccaaatacgtctgtgctgctcagtcggggccgtattgttatcgtccccttcacccgtcggaatataacaaatggggtcaggggacccaggtcaccgtctcctcagcggccgcggcacaccatcaccaccatcatggcgcagaacaaaaactcatctcagaagaggatctgtcttag

SEQ ID NO: 130 - DNA, 432 bp, synthetic construct
  source 1..432: mol_type = other DNA
gatgtgcagctgcaggagtctgggggaggctcggtggaggctggaggatctctgacactcacctgtgtagcctctgaatttaggtccatgatggcatggtatcgtcaggccccagggaaggagtgcgaatttctctctcggattcctagtgatggtgtccctgtgtatggcgactccatgaagggccgatcgagcatctcccgagacaacgccaagaatacggtgacccttcagttgaacagtctgaaagttgaggatacggccatgtattactgcgcatcaacctgggcggcctccggaggtaattgccctaagccttgggattactggggccaggggacccaggtcaccgtctcctcagcggccgcggcacaccatcaccaccatcatggcgcagaacaaaaactcatctcagaagaggatctgtcttag

SEQ ID NO: 131 - protein, 13 aa, synthetic construct
GYTRGTFRSNCMG

SEQ ID NO: 132 - protein, 10 aa, synthetic construct
AIYTGEGNTY

SEQ ID NO: 133 - protein, 17 aa, synthetic construct
RGGYCSDTYWRWDDYNY

SEQ ID NO: 134 - protein, 6 aa, synthetic construct
DSKNTM

SEQ ID NO: 135 - protein, 9 aa, synthetic construct
RVTSRDTMA

SEQ ID NO: 136 - protein, 10 aa, synthetic construct
TVYPGTAETY

SEQ ID NO: 137 - protein, 19 aa, synthetic construct
AARRAVFGRIALEDENYSD

SEQ ID NO: 138 - protein, 6 aa, synthetic construct
DKTKNM

SEQ ID NO: 139 - protein, 10 aa, synthetic construct
GATYCTGDMS

SEQ ID NO: 140 - protein, 13 aa, synthetic construct
TISSDGSTYYADS

SEQ ID NO: 141 - protein, 17 aa, synthetic construct
QSGPYCYRPLHPSEYNK

SEQ ID NO: 142 - protein, 6 aa, synthetic construct
DNATIY

SEQ ID NO: 143 - protein, 7 aa, synthetic construct
EFRSMMA

SEQ ID NO: 144 - protein, 13 aa, synthetic construct
RIPSDGVPVYGDS

SEQ ID NO: 145 - protein, 15 aa, synthetic construct
TWAASGGNCPKPWDY